(19) United States
(12) Patent Application Publication
(10) Pub. No.: US 2023/0313281 A1
(43) Pub. Date: Oct. 5, 2023

(54) Methods and Compositions For Preparing Nucleic Acids For Genetic Analysis

(71) Applicant: Takara Bio USA, Inc., San Jose, CA (US)

(72) Inventors: Jacob Meyers, San Jose, CA (US); Julie Catherine Laliberté, San Jose, CA (US); Patrick Kevin Martin, San Jose, CA (US)

(21) Appl. No.: 18/022,844

(22) PCT Filed: Sep. 28, 2021
(86) PCT No.: PCT/US2021/052337

§ 371 Date: Feb. 23, 2023

Related U.S. Application Data
(60) Provisional application No. 63/089,719, filed on Oct. 9, 2020.

(51) Int. Cl. C12Q 1/6851; C12Q 1/6806

(52) U.S. Cl. CPC C12Q 1/6851; C12Q 1/6806; C12Q 2600/156

(57) ABSTRACT

The present disclosure provides methods and compositions for preparing nucleic acid molecules using a novel, single-tube workflow that allows users to carry out the isolation of nucleic acids from a sample and perform a nucleic acid assay using the isolated nucleic acids in a single tube or vessel. As such, the methods disclosed herein can be used with low quality and/or small quantity of nucleic acid samples. Further, the disclosed methods offer similar or improved concordance rates in addition to improved signal to noise ratio when compared to the existing methods used for genetic analysis. Thus, making the methods disclosed herein suitable for clinical applications such as infectious disease diagnosis, pre-implantation genetic testing, prenatal or cancer diagnosis.

### CROSS-REFERENCE TO RELATED APPLICATIONS

Pursuant to 35 U.S.C. § 119(e), this application claims priority to the filing date of the U.S. Provisional Patent Application Ser. No. 63/089,719, filed Oct. 9, 2020, the disclosure of which application is herein incorporated by reference.

### INTRODUCTION

Recently, workflows for preparing nucleic acid molecules for using them in clinical applications, for example, for performing genetic analysis, have been developed. These methods require the processing of samples using multi-step protocols requiring multiple transfers of samples into different containers, causing substantial sample loss during the processing of samples. Hence, the existing workflows may not be efficiently used with low quality and/or small quantity of nucleic acid samples, for example, cell-free nucleic acids in spent media or culture media.

### SUMMARY

The present disclosure provides methods and compositions for preparing nucleic acid molecules using a novel, single-tube workflow that allows users to carry out the isolation of nucleic acids from a sample and perform a nucleic acid assay using the isolated nucleic acids in a single tube or vessel. As such, the methods disclosed herein can be used with low quality and/or small quantity of nucleic acid samples. Further, the disclosed methods offer similar or improved concordance rates in addition to improved signal to noise ratio when compared to the existing methods used for genetic analysis. Thus, making the methods disclosed herein suitable for clinical applications such as infectious disease diagnosis, pre-implantation genetic testing, prenatal or cancer diagnosis.

In an aspect, the present disclosure provides a method of preparing nucleic acid molecules from a sample. The method comprises optionally lysing the sample to release the nucleic acid molecules, isolating the released nucleic acid molecules using a plurality of beads for binding of the nucleic acid molecules to the plurality of beads, and eluting the bound nucleic acid molecules from the plurality of beads by adding one or more reagents for performing a nucleic acid assay.

In some embodiments, the method further comprises performing the steps in a single vessel. In some embodiments, one or more of steps a-c are performed simultaneously. In some embodiments, the sample is lysed using a lysis reagent, where lysis reagents that may be employed include, but are not limited to, detergents, for example Tween 20, Triton X 100, SDS and other detergents, e.g., as known in the art, proteases, such as proteinase K, thermolysin, trypsin, and the like, etc. where in some embodiments, the lysis reagent is proteinase K.

In some embodiments, the nucleic acid assay is a polymerase chain reaction, whole genome amplification, whole transcriptome amplification, sequencing, or any combination thereof. In some embodiments, the nucleic acid assay is performed for determining at least one genetic variant. In some embodiments, the at least one genetic variant is aneuploidy, mosaicism, single nucleotide polymorphism, and any combination thereof. In some embodiments, the nucleic acid molecules are selected from the group consisting of DNA, RNA, or cDNA.

In some embodiments, the plurality of beads are magnetic beads. A variety of magnetic beads are known in the art that are suitable for practicing embodiments of the invention including, but not limited to: Macharey Nagel p-beads, Omega Bio-tek Mag-Bind TotalPure NGS beads, AMPure XP beads, NucleoMag NGS Clean-up and Size Select beads from Takara Bio, Inc., as well as the beads provided in the MiniMax High Efficiency cfDNA Isolation Kit and the MagVigen Plasma cfDNA Extraction Kit. In some embodiments, the magnetic beads are p-beads. In some embodiments, the sample is selected from the group consisting of blood, whole blood, plasma, serum, urine, cerebrospinal fluid, feces, saliva, sweat, tears, pleural fluid, pericardial fluid, peritoneal fluid, semen, uterine lavage, breast milk, extracellular vesicles, culture media, somatic cells, germ cells, fetal cells, pap smear, maternal cells, and environmental sample.

### BRIEF DESCRIPTION OF THE FIGS.

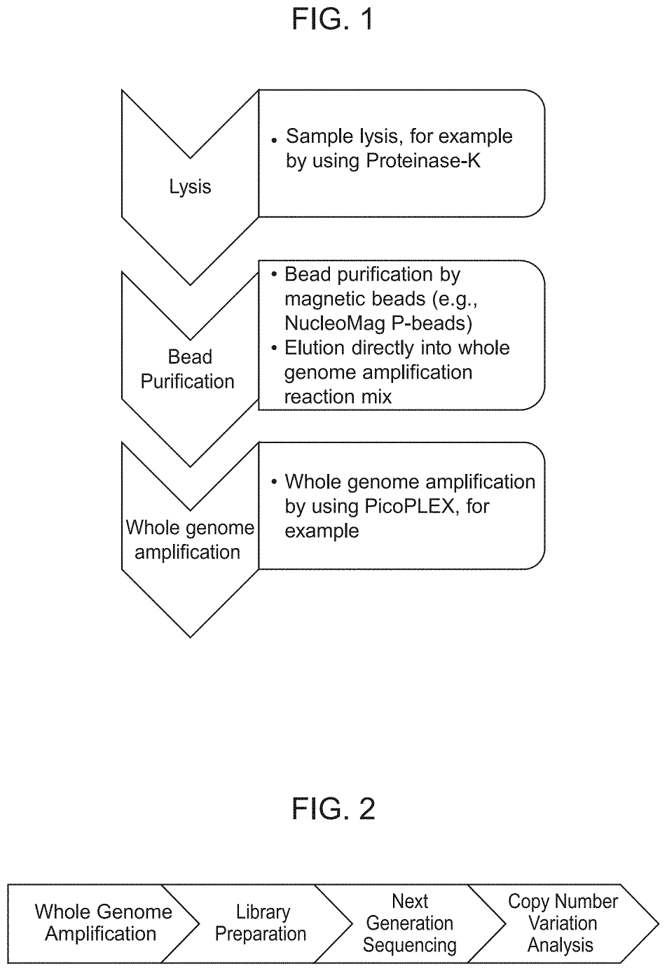

FIG. 1 depicts the main steps of a single-tube workflow that combines nucleic acid (e.g., DNA) isolation and whole genome amplification (WGA) in a single tube.

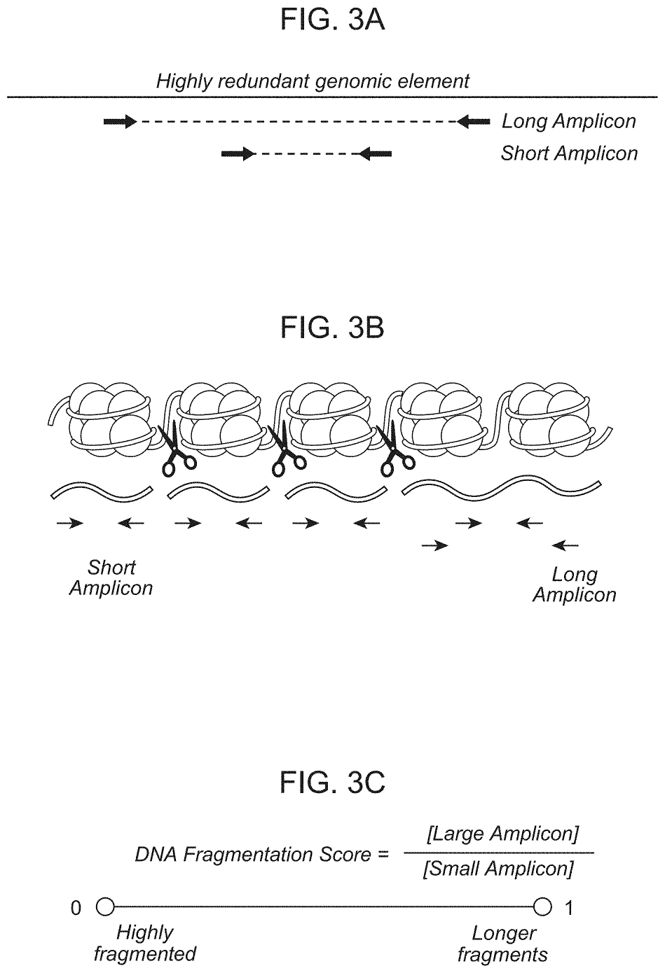

FIG. 2 shows the steps of a CNV or aneuploid detection workflow. FIG. 3 shows the relative positions of primers and amplicons for determining the quality of DNA using the DNA Fragmentation Score. FIG. 3A shows the relative positions of long and short amplicons on a highly redundant target genomic element. FIG. 3B shows the positions of long and short primers relative to the positions of nucleosomes in a genome. FIG. 3C shows the qualitative assessment of DNA using the DNA Fragmentation Score.

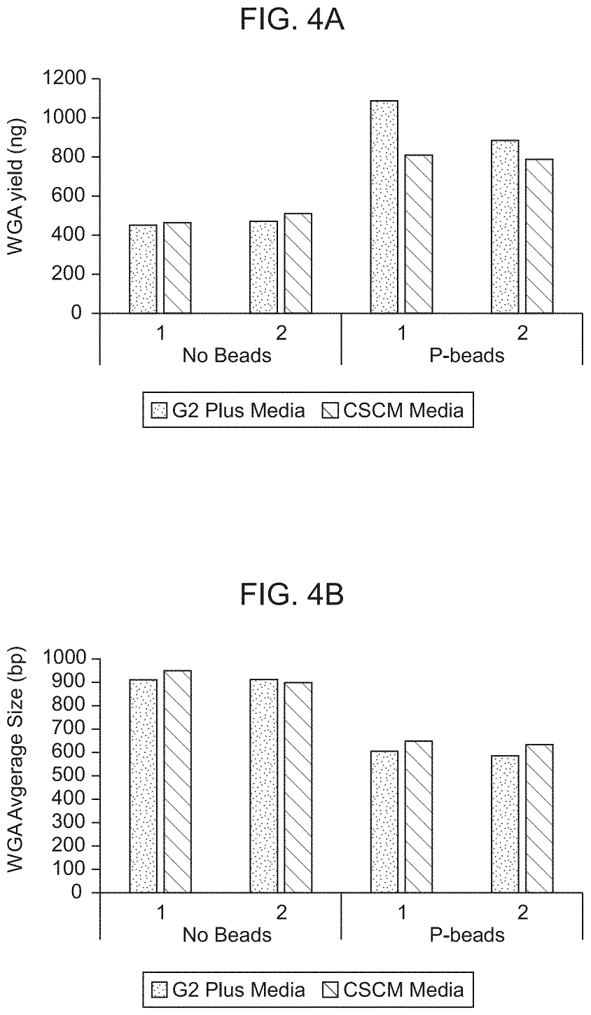

FIG. 4 shows the comparison of WGA yield and WGA product size using the standard PicoPLEX WGA workflow with a novel single-tube workflow disclosed herein. FIG. 4A shows the WGA yield from the standard PicoPLEX workflow and the new workflow using cell-free DNA from human embryonic stem cells cultured in two different culture media. FIG. 4B shows the average size of WGA product from the standard PicoPLEX workflow and the new workflow.

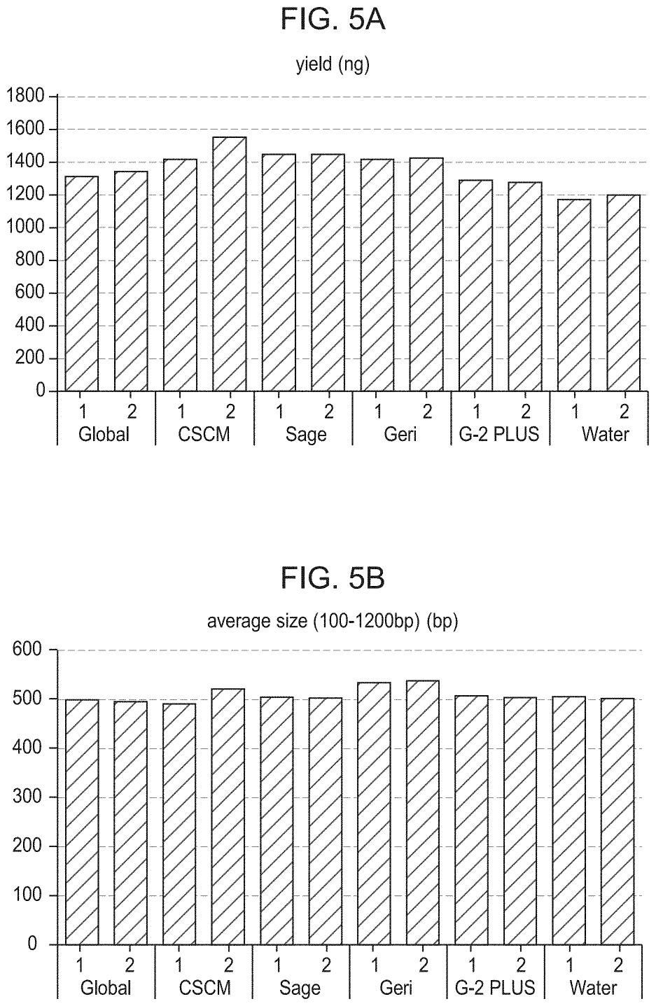

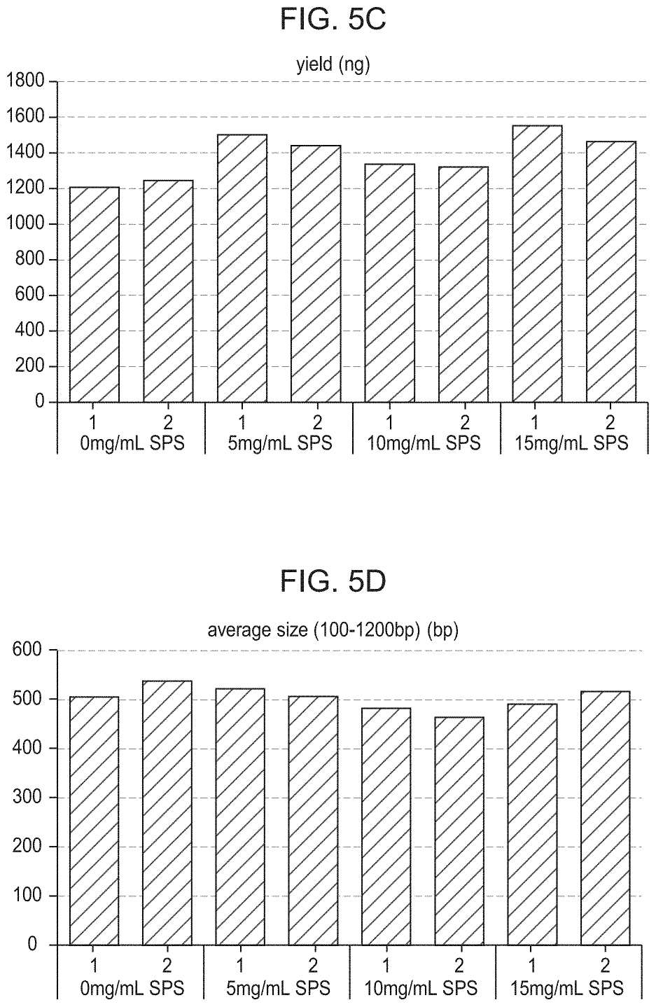

FIG. 5 shows the WGA product yield and average WGA product size as obtained using a novel single-tube workflow as disclosed herein performed with 1.5 kb sheared gDNA diluted in different culture media and culture media supplements. (FIG. 5A) shows the WGA yield from gDNA diluted in five embryo media products and water. (FIG. 5B) shows the WGA product average size from gDNA diluted in five embryo media products and water. (FIG. 5C) shows the WGA yield from gDNA diluted in Global media containing either 0 mg/ml, 5 mg/ml, 10 mg/ml or 15 mg/ml of serum protein substitute (SPS). (FIG. 5D) shows the WGA yield from gDNA diluted in Global media containing either 0 mg/ml, 5 mg/ml, 10 mg/ml or 15 mg/ml of SPS.

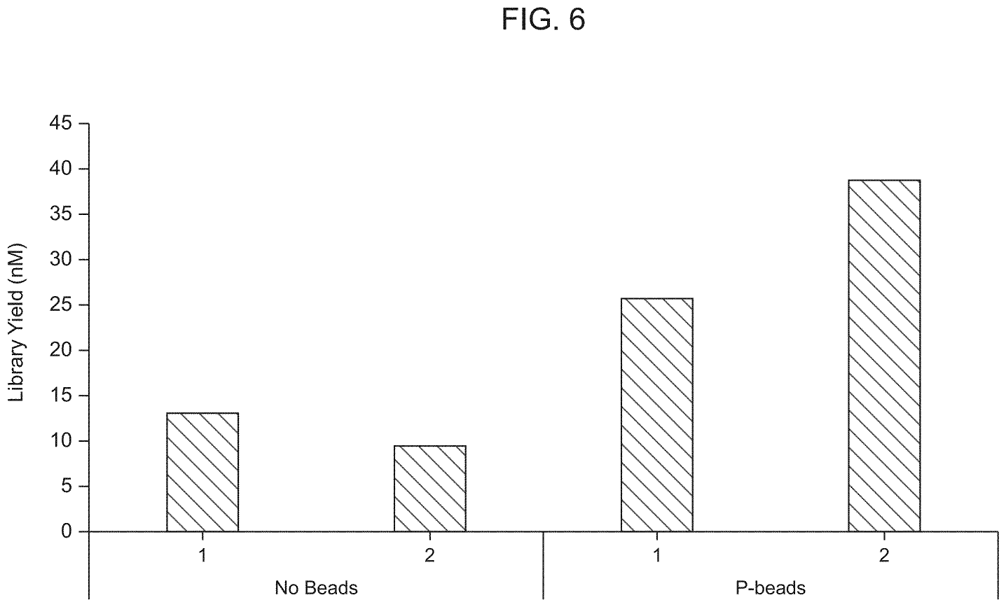

FIG. 6 shows the NGS library yield from the standard PicoPLEX workflow and a novel single-tube workflow as disclosed herein.

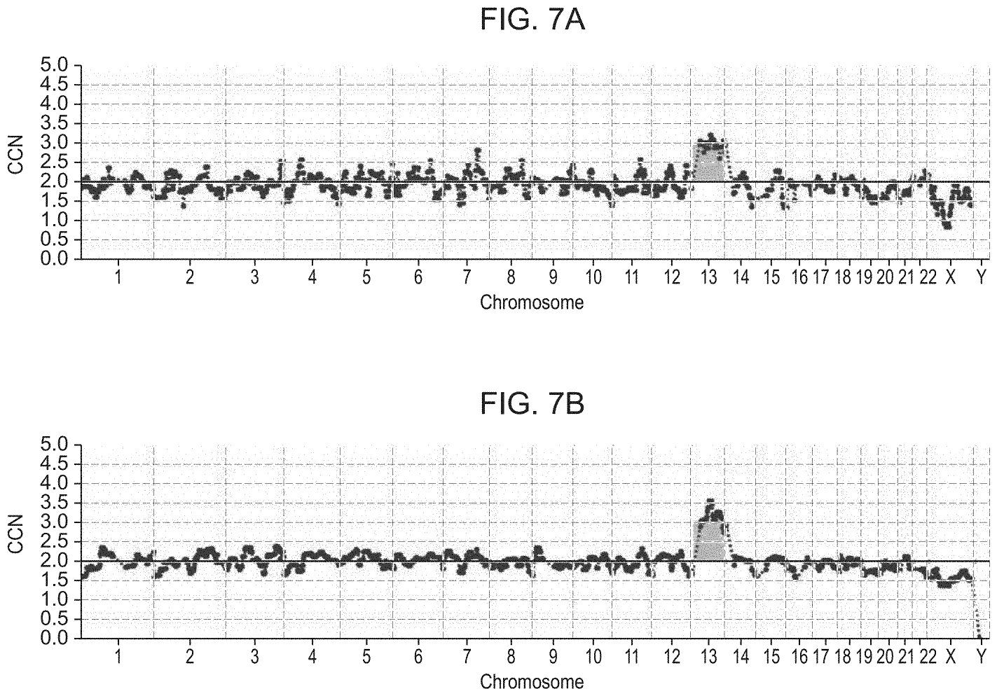

FIG. 7 shows the CNV plots, depicting chromosomal abnormalities, from the standard PicoPLEX workflow (FIG. 7A) and a novel single-tube workflow (FIG. 7B).

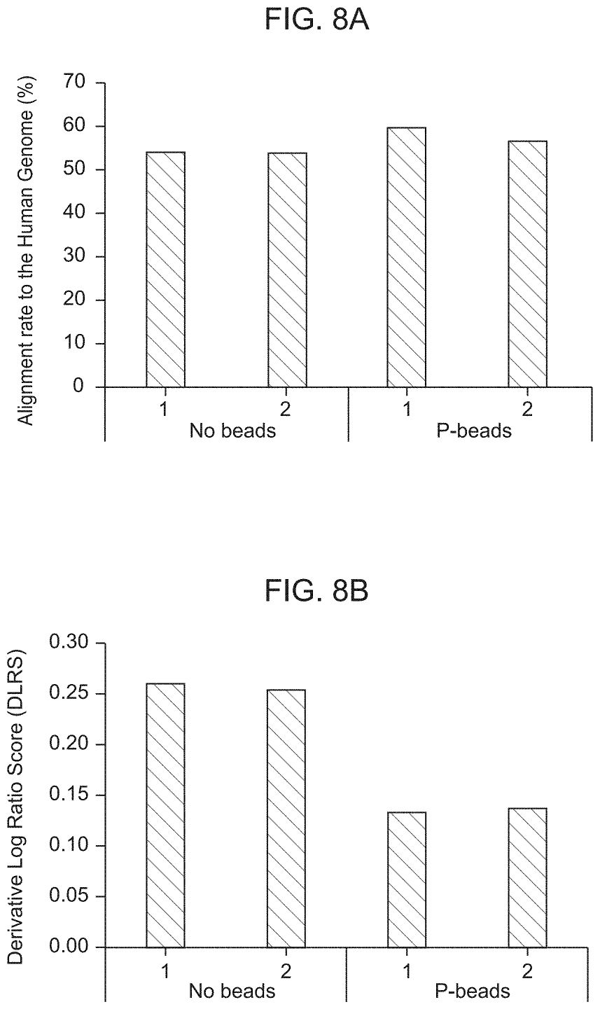

FIG. 8 shows the comparative performance of the standard PicoPLEX workflow and a novel single-tube workflow in terms of genomic alignment of sequencing reads and signal noise in the generated copy number profiles. FIG. 8A shows the alignment rate of the sequencing reads generated from the two workflows while FIG. 8B shows the CNV noise expressed as Derivative Log Ratio Score (DLRS) between the two workflows.

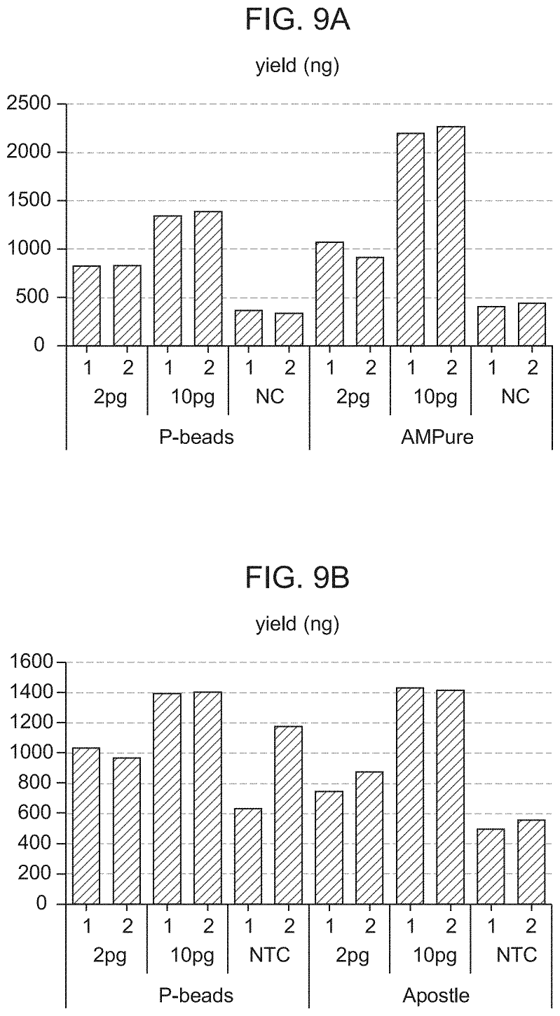

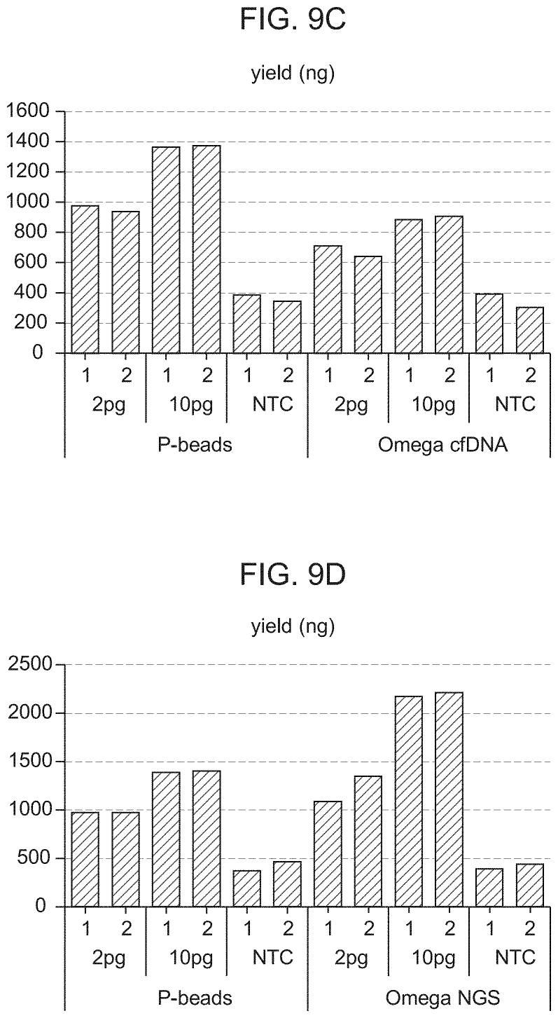

FIG. 9 shows a comparison of a single-tube workflow in accordance with embodiments of the invention using various bead products compared to P-beads using either a 2 pg or 10 pg sheared gDNA input as well as a negative control (NTC). (FIG. 9A) Shows the WGA yield of P-beads vs AMPure XP beads. (FIG. 9B) Shows the WGA yield of P-beads vs the Apostle MiniMax High Efficiency Isolation Kit. (FIG. 9C) Shows the WGA yield of P-beads vs Mag-Bind TotalPure NGS (FIG. 9D) Shows the WGA yield of P-beads vs Mag-Bind cfDNA Kit (Omega Beads).

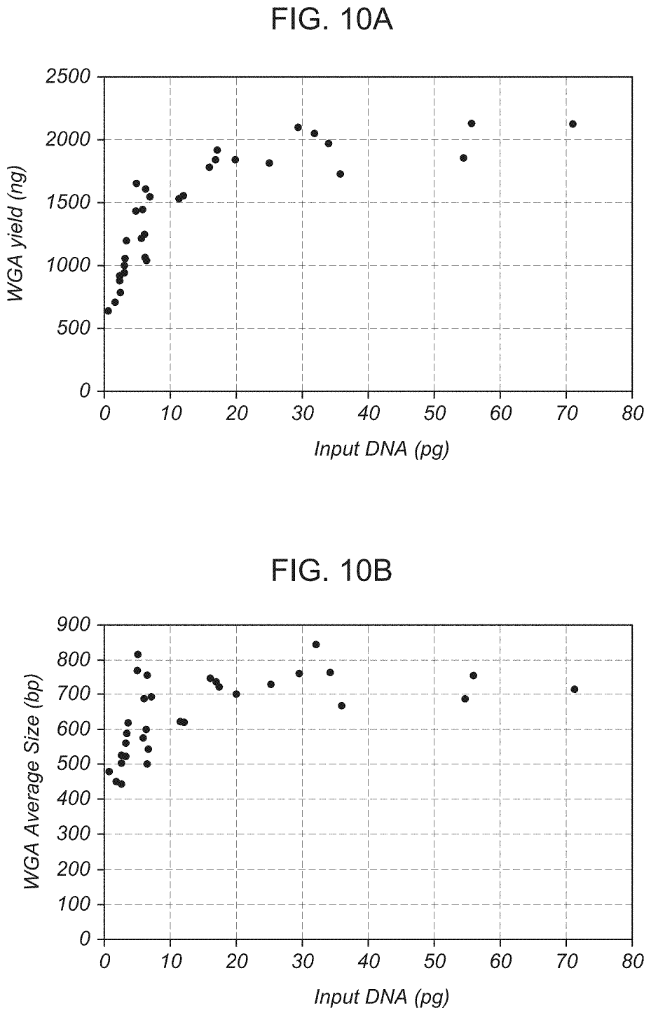

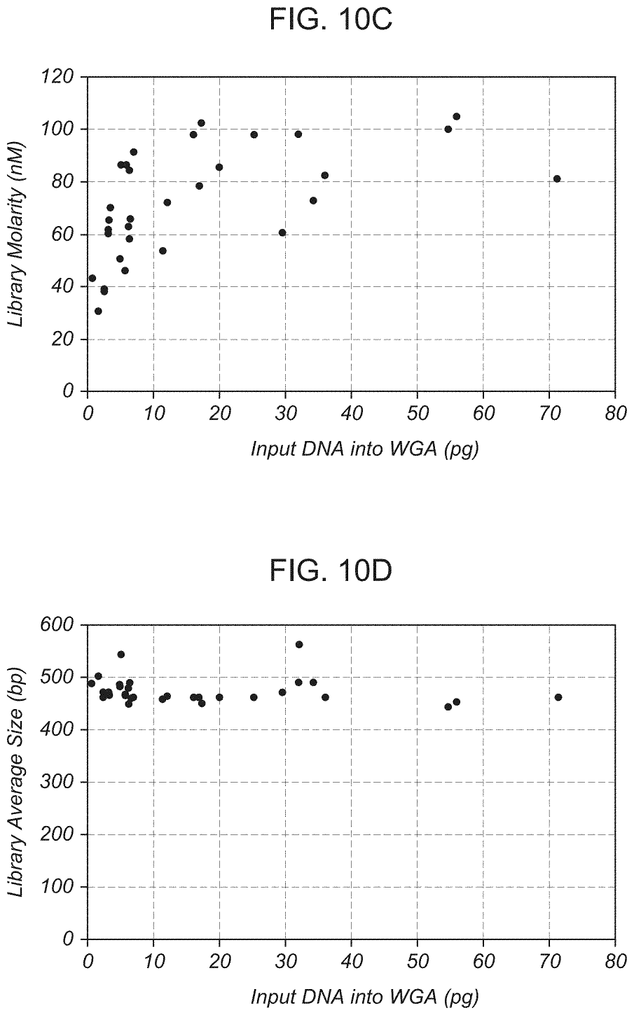

FIG. 10 shows the relationship between the WGA yield (FIG. 10A), average size of the WGA product (FIG. 10B), NGS library yield (FIG. 100), or the average size of the NGS library (FIG. 10D) to input cell-free DNA amount in spent media samples using a single-tube workflow disclosed herein.

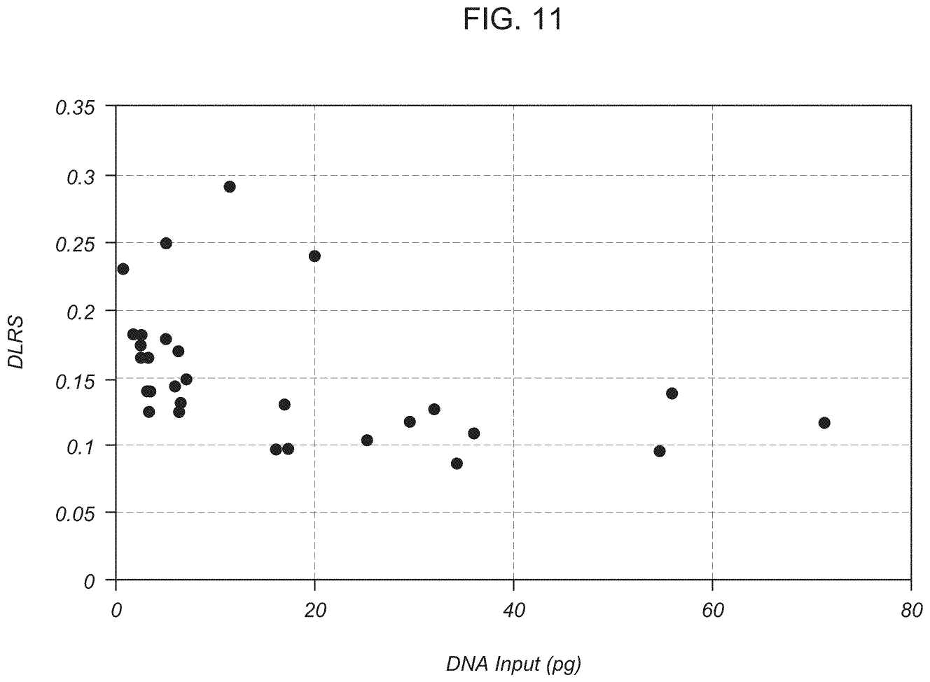

FIG. 11 shows the relationship between CCN noise, expressed as Derivative Log Ratio Score (DLRS), to input cell-free DNA amount in spent media using the single-tube workflow along with the CNV detection workflow disclosed herein.

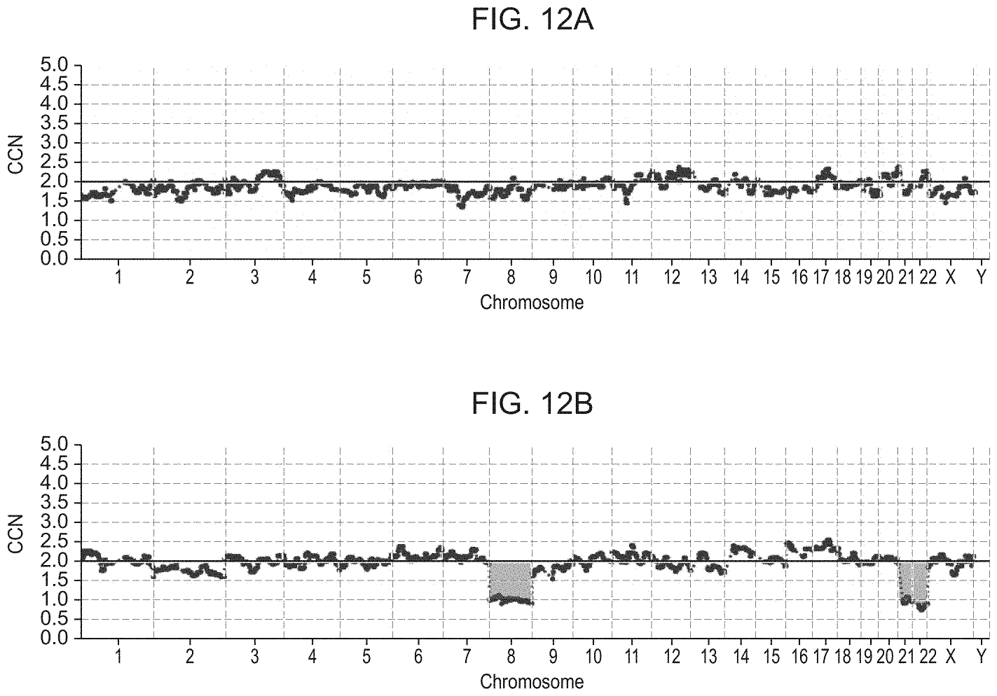

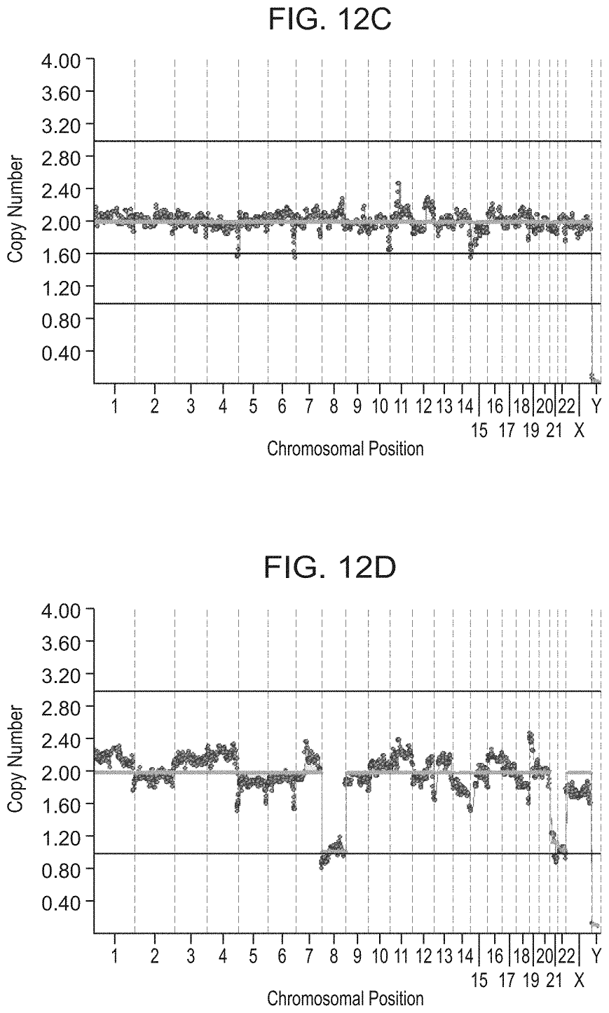

FIG. 12 shows a comparison between CNV plots generated using the Takara Bio USA Analysis software and using a single-tube workflow in accordance with an embodiment of the invention on spent culture media from embryo samples (FIG. 12A and FIG. 12B) to data obtained using the BlueFuse Multi Analysis Software and the VeriSeq chemistry on trophectoderm biopsy samples of the same embryo sample (FIG. 12C and FIG. 12D) for a euploid (FIG. 12A and FIG. 12C) and an aneuploid (FIG. 12B and FIG. 12D) sample.

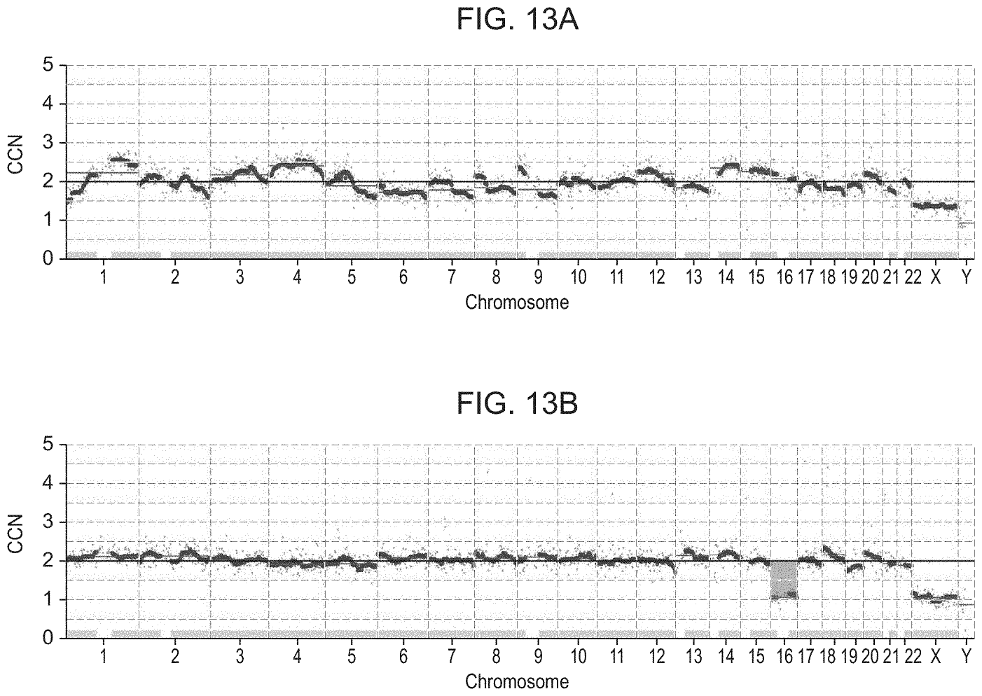

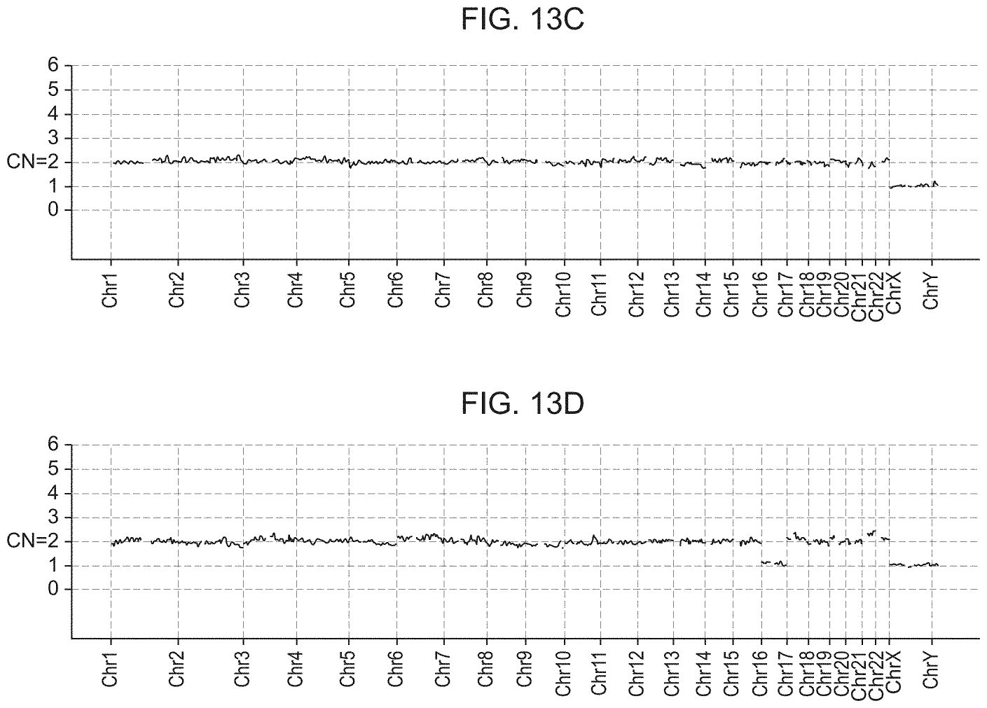

FIG. 13 shows the comparison between the CNV plots generated using the Takara Bio USA Analysis software and using a novel single-tube workflow in accordance with an embodiment of the invention on spent culture media from embryo samples (FIG. 13A and FIG. 13B) to data obtained using an alternative software tool and VeriSeq Chemistry on trophectoderm biopsy samples from the same embryo sample (FIG. 13C and FIG. 13D) for a euploid (FIG. 13A and FIG. 13C) and aneuploid (FIG. 13B and FIG. 13D) sample. The embryo sample used in generating this data in FIG. 13 was obtained from a different IVF clinic than the embryo sample shown in FIG. 12.

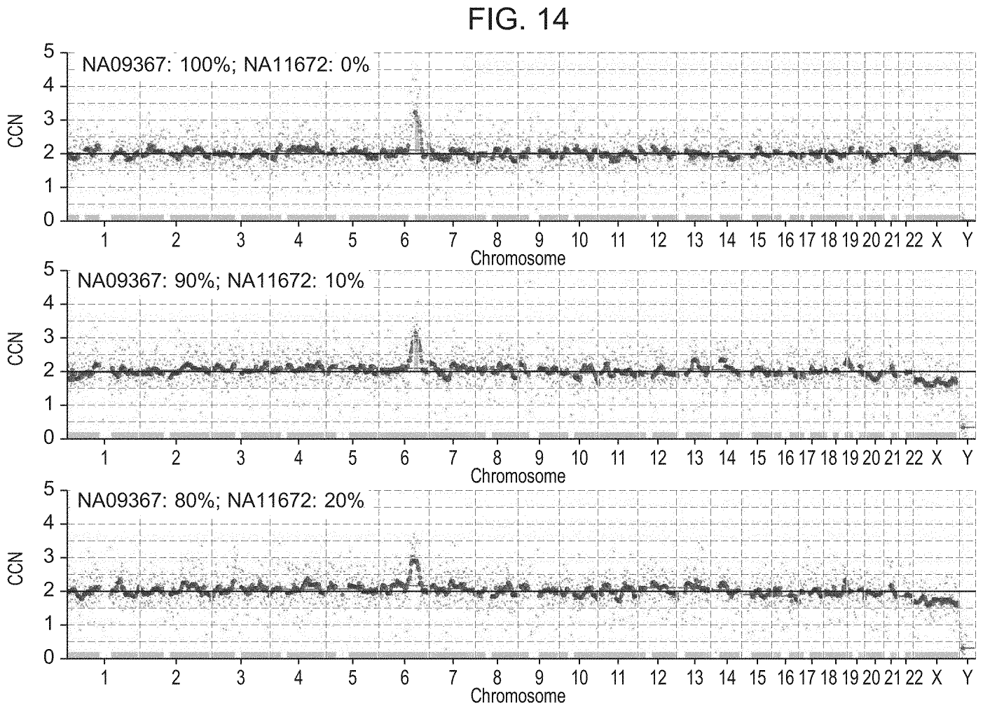

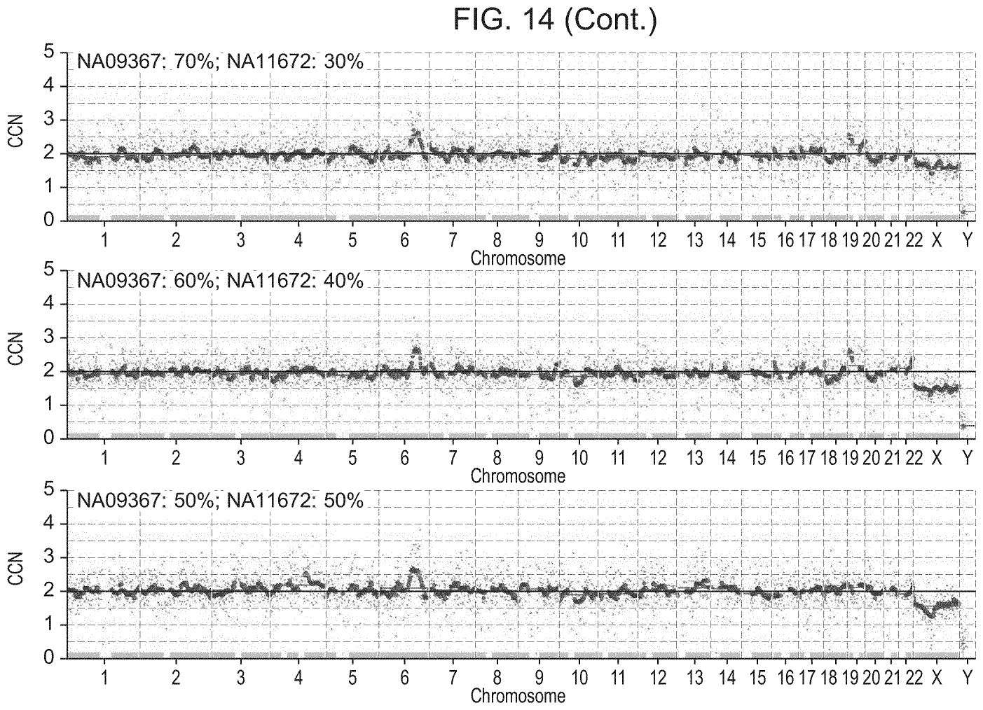

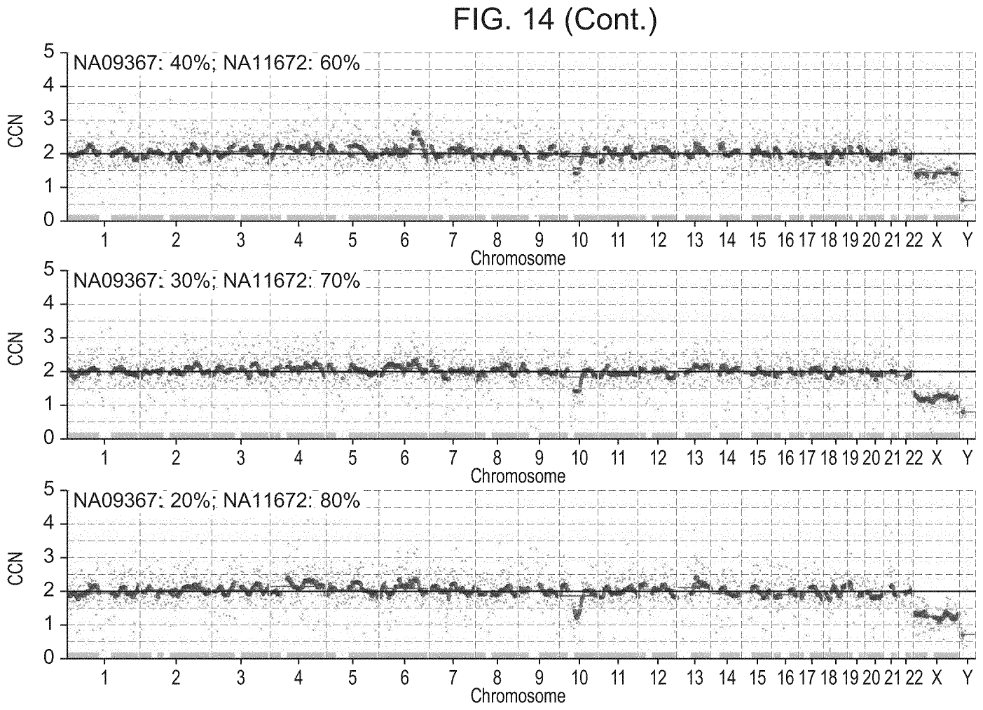

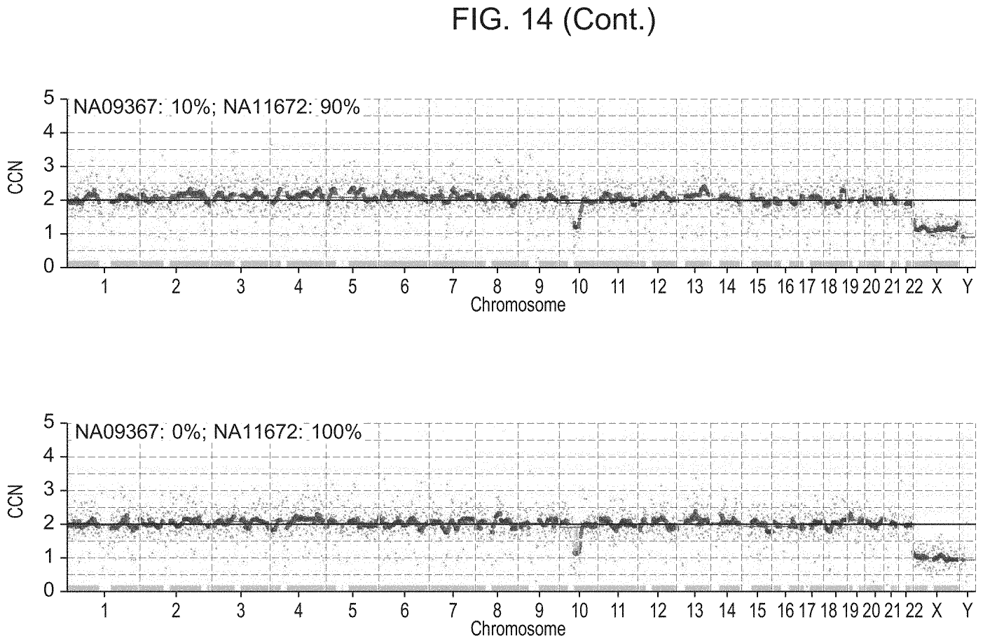

FIG. 14 shows the CNV plots from mosaic genomic DNA mixtures of NA09367: 46 XX, +6: 35.2 MB, and NA11672 46 XY, −10: 26.2 MB

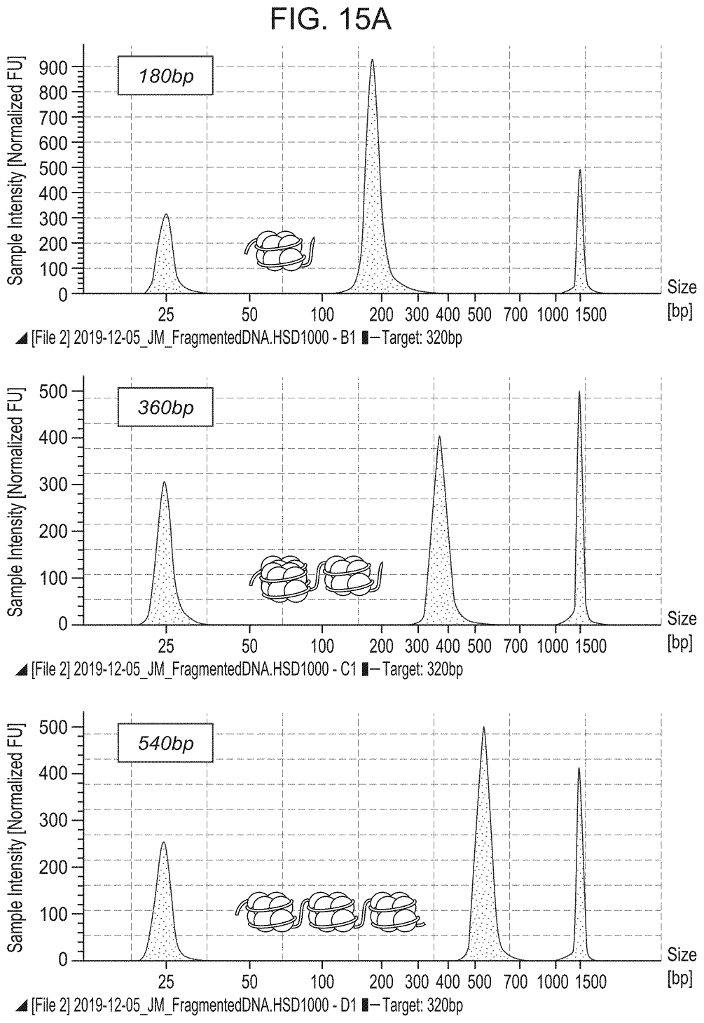

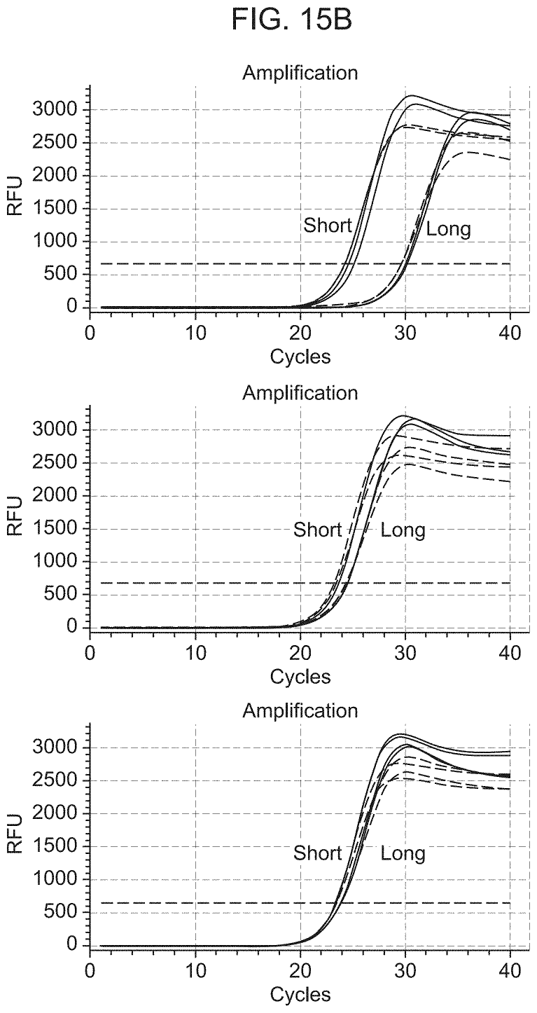

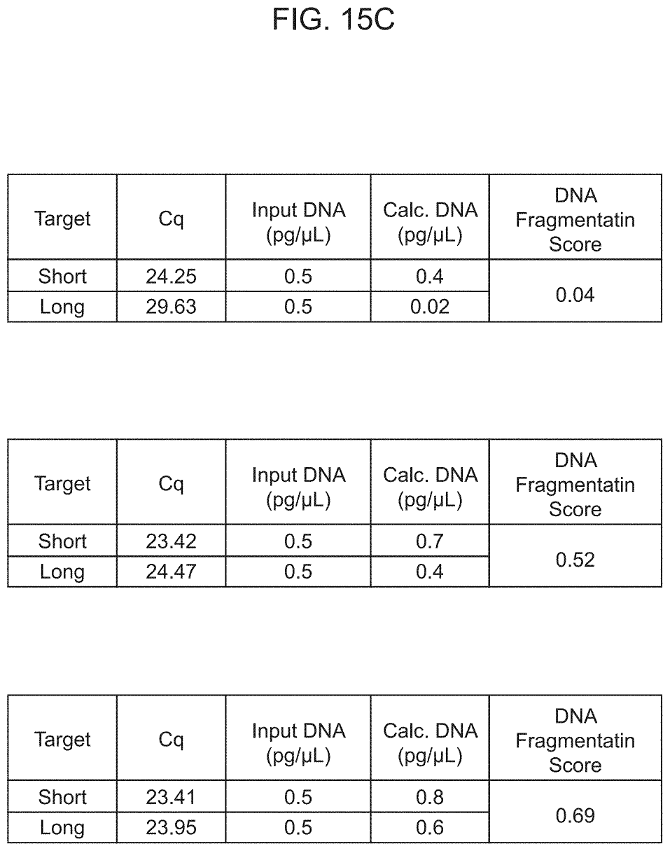

FIG. 15 shows the qualitative and quantitative assessment of sheared, size-selected DNA. FIG. 15A shows the size profile of the sheared DNA. FIG. 15B shows the amplification of the sheared DNA using the short and long primer pairs. FIG. 15C shows the DNA quantity and quality, expressed as the DNA Fragmentation Score, compared to the input DNA.

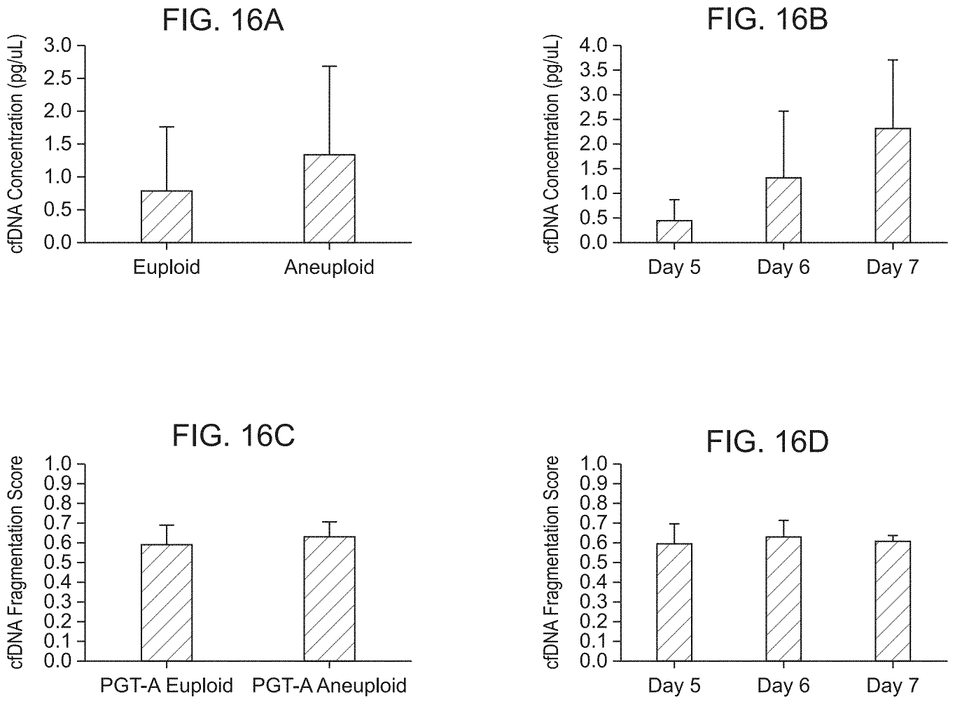

FIG. 16 shows the qualitative and quantitative assessment of cell-free DNA in spent media, collected from spent media collected from Day 5 to Day 7, obtained using the qPCR method described in FIG. 3. FIG. 16A and FIG. 16B show the concentration of cfDNA in the medium and FIG. 16C and FIG. 16D show the fragmentation score. In FIG. 16A and FIG. 16C, the concentration and fragmentation score are shown in relation to the status of the embryo—either Euploid or Aneuploid and in FIG. 16B and FIG. 16D, the concentration and fragmentation score are shown in relation to the age of the blastocyst at the time the culture medium was collected.

### DETAILED DESCRIPTION

Recently, workflows for preparing nucleic acid molecules for using them in clinical applications, for example, for performing genetic analysis, have been developed. These methods require the processing of samples using multi-step protocols requiring multiple transfers of samples into different containers, causing substantial sample loss during the processing of samples. Hence, the existing workflows may not be efficiently used with low quality and/or small quantity of nucleic acid samples, for example, cell-free nucleic acids in spent media or culture media.

The present disclosure provides methods and compositions for preparing nucleic acid molecules using a novel, single-tube workflow that allows users to carry out the isolation of nucleic acids from a sample and perform a nucleic acid assay using the isolated nucleic acids in a single tube or vessel. As such, the methods disclosed herein can be used with low quality and/or small quantity of nucleic acid samples. Further, the disclosed methods offer similar or improved concordance rates in addition to improved signal to noise ratio when compared to the existing methods used for genetic analysis. Thus, making the methods disclosed herein suitable for clinical applications such as infectious disease diagnosis, pre-implantation genetic testing, prenatal or cancer diagnosis.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods

The methods disclosed herein can be used with nucleic acids isolated from a variety of samples, such as blood, whole blood, plasma, serum, urine, cerebrospinal fluid, feces, saliva, sweat, tears, pleural fluid, pericardial fluid, peritoneal fluid, semen, uterine lavage, breast milk, extracellular vesicles, culture media, somatic cells, germ cells, fetal cells, pap smear, or maternal cells, environmental samples, samples found at crime scenes, or water samples. The samples can be obtained from healthy or diseased individuals. In some cases, the samples can be obtained from an embryo, such as trophectoderm biopsy. In some cases, the samples can be derived from a tumor biopsy or fine needle aspirate. In some cases, the samples can be obtained from a cultured cell or cells, organelles, organs, etc. In some cases, the samples can be preserved, such as FFPE samples.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

The nucleic acid molecules isolated from a sample can be cell-free nucleic acids comprising cell-free deoxyribonucleic acid (cfDNA). In some embodiments, the cell-free nucleic acids comprise cell-free ribonucleic acid (cfRNA). Cell-free nucleic acids can refer to nucleic acid molecules that can be found outside cells, in bodily fluids such as blood, whole blood, plasma, serum, urine, cerebrospinal fluid, fecal, saliva, sweat, tears, pleural fluid, pericardial fluid, peritoneal fluid, semen, uterine lavage, or breast milk of a subject. Cell-free nucleic acids can also be found in cell culture media or spent media, extracellular vesicles, etc. Cell-free nucleic acids may originate from one or more healthy cells, fetal cells, maternal cells, and/or from one or more cancer cells. Examples of the cell-free nucleic acids include but are not limited to RNA, mitochondrial DNA, or genomic DNA. Cell-free nucleic acids, where desired, may be diluted in a suitable culture medium, where suitable culture media include, but are not limited to standard human embryo culture media, such as G-2 PLUS media (Vitrolife), Global+Quinn's Advantage Serum Substitute, Sage 1-Step (Vitrolife), Geri Medium (Geri), Continuous Single Culture Complete with HSA media (CSCM) (Irvine Scientific), MEM, DMEM, RPMI, Ham's F12 and the like. Culture media may additionally contain serum, such as fetal bovine serum, or serum substitutes including but not limited to human serum albumin (HSA), bovine serum albumin (BSA), serum protein substitute (SPS) such as available from Irvine Scientific or Quinn's Advantage Serum protein Substitute and the like.

In some cases, the methods disclosed herein allow users to input a larger sample volume compared with the existing methods. In some embodiments, the input volume of a sample comprising nucleic acid can be between 1 μl to 1 ml. In some embodiments, the sample input volume can be between 5 μl to 500 μl. In some embodiments, the sample input volume can be between 50 μl to 400 μl. In some embodiments, the sample input volume can be between 100 μl to 300 μl.

The methods disclosed herein can be carried out in a single vessel, for example, a tube, well, or cavity. In some cases, some steps can be carried out in a first vessel while the rest can be carried out in a second vessel. In some cases, the first vessel and the second vessel can be connected. For example, the first vessel can be a column with an inlet and an outlet, and the outlet can be fluidically connected to the second vessel. In this case, the second vessel has an opening, connected to the first vessel, and a closed end. In some embodiments, the first and second vessels can be disconnected.

The methods disclosed allow for carrying out the nucleic acid isolation procedure and nucleic acid assay in a single tube or vessel. The nucleic acids can be isolated using an optional lysis step employing a lysis reagent, e.g., a protease, such as proteinase K, for lysing the sample to release nucleic acids in solution. As reviewed above, any convenience lysis agent may be employed in this step, where lysis reagents that may be employed include, but are not limited to, detergents, for example Tween 20, Triton X 100, SDS and other detergents, e.g., as known in the art, proteases, such as proteinase K, thermolysin, Trypsin and the like, etc. The released nucleic acids can be isolated by providing beads or particles for the nucleic acids to bind to. Such beads or particles can be magnetic beads, for example. The magnetic beads can be functionalized to bind to specific nucleic acid molecules in the sample. For example, a magnetic bead surface can be functionalized with carboxylate moiety for the capture of biomolecules by non-covalent attachment, thus can be used for affinity purification and pull-down. In some cases, the magnetic bead surface can be functionalized with amine moiety for purification of biomolecules. In some other cases, the magnetic bead surface can be functionalized to bind to specific nucleic acid sequences, such as oligo(dT), that could hybridize to the complementary sequences in a sample. In some cases, the beads can size-select nucleic acid molecules. In some cases, beads can be used to isolate various types of biomolecules, such as genomic DNA, plasmids, mitochondrial DNA, RNA, and/or proteins. In some cases, beads can be functionalized with streptavidin moiety for high-specificity biotin-binding applications. In some cases, beads can be functionalized with Protein A/G for the isolation of specific antibodies. In some cases, beads can be functionalized with silica for applications requiring reversible binding of nucleic acids. In some other cases, beads can be functionalized with a porous material, such as sepharose, for affinity purification.

After binding nucleic acid molecules to the beads, an external magnetic field can be used to attract the beads to the outer edge of the containing tube, immobilizing them. While the beads are immobilized, the bead-bound nucleic acid molecules can be retained during the washing steps. In some cases, one washing step may be used while in other cases multiple washing steps may be used depending on the purity of the sample or the contaminants present in the sample. After the washing step(s), the bead-bound nucleic acid can be eluted by removing the magnetic field then releasing the nucleic acid molecules as a purified sample, ready for quantitation and analysis. In an alternative embodiment, the bead-bound nucleic acid is not eluted from the beads, but instead is treated directly with reagents for a nucleic acid assay, e.g., such as described in more detail below.

The purified nucleic acid molecules can be used for a nucleic acid assay, for example, whole genome amplification. In some cases, the reagents for nucleic acid assay(s) can directly be added to the tube containing the purified nucleic acid molecules, i.e., eluted from the magnetic beads, and the assay can be carried out in the same tube, thus making it a single-tube workflow.

In this case, the purified nucleic acid molecules are in solution. In some cases, the reagents for a nucleic acid assay(s) can directly be added to the tube containing the nucleic acid molecules while they are still bound to the magnetic beads, and the assay(s) can be carried out on the bound nucleic acid molecules. In some other cases, some nucleic acid molecules are bound to the magnetic beads while some are in solution. In some cases, the reagents for a single assay can be added to the tube containing the purified nucleic acid molecules. In other cases, multiple assays can be carried out by adding the reagents required for those assays to the tube. In some cases, different reagents can be added at once to carry out assays simultaneously. In some other cases, different reagents can be added one after the other. In some cases, some reagents can be added at once while some can be added one after the other.

The purified nucleic acid molecules can be subjected to nucleic acid assay(s) including, but not limited to, whole genome amplification, whole transcriptome amplification, reverse transcription, polymerase chain reaction, sequencing, etc. Targeted amplification or other methods of targeted enrichment may also be employed either separately or in concert with methods of whole genome amplification or whole transcriptome amplification to focus on specific chromosomes or regions known to harbor particular variations of interest. For example, methods such as those disclosed in U.S. Provisional Patent Application Ser. No. 62/806,698 may be employed in the present invention to obtain both copy number variation and single nucleotide polymorphism analysis from the same sample. The results from the assay(s) can further be used to perform genetic analysis. In some embodiments, the analysis can include determining the quantity of the nucleic acid molecules. For example, the results from a quantitative PCR can be used to determine the quantity of nucleic acid molecules in a sample. The quality of the nucleic acid—for example, the degree of fragmentation—may also be assessed as discussed further below.

In some embodiments, the analysis can include assessing the quality of the nucleic acid molecules. This disclosure provides a method to determine the quality by dividing the quantity of high-integrity nucleic acid molecules by the total amount of nucleic acid molecules in a sample. For example, the quality of DNA can be determined by quantifying the amounts of a short genomic region and a long genomic region to produce the short and the long amplicon, respectively using a quantitative PCR assay. Then, a ratio of the large amplicon to the short amplicon is used to obtain the DNA Fragmentation Score, indicative of the quality of DNA. The ratio varies from 0-1, with values approaching zero indicating greater degrees of DNA fragmentation in the sample.

$$DNA Fragmentation Score = \frac{[ Large Amplicon ]}{[ Short Amplicon ]}$$

In some embodiments, the analysis can include determining genetic variant(s) or variance that may occur in the nucleic acid molecules in a sample. In certain embodiments, the presence or absence of one or more genetic variations is determined according to an outcome provided by the methods described herein. A genetic variation can be a particular genetic phenotype present in certain individuals, and often a genetic variation is present in a statisticaliy significant sub-population of individuals. In some embodiments, a genetic variation can be a chromosome abnormality (e.g., aneuploidy), partial chromosome abnormality, or mosaicism, each of which is described in greater detail herein. Non-limiting examples of genetic variations include one or more deletions (e.g., micro-deletions), duplications (e.g., micro-duplications), insertions, mutations, polymorphisms (e.g,, single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (e.g., about 10 bp, 50 bp, 100 bp, 500 bp, 1 kb, 5 kb, 10 kb, 50 kb, 100 kb, 500 kb, or 1000 Kb in length).

A genetic variation can be a deletion. A deletion can be a mutation (e.g., a genetic aberration) in which a part of a chromosome or a sequence of DNA is missing. A deletion represents the loss of genetic material from a particular position in the genome. Any number of nucleotides can be deleted. A deletion can comprise the deletion of one or more entire chromosomes, a segment of a chromosome, an allele, a gene, an intron, an axon, any non-coding region, any coding region, a segment thereof or combination thereof. A deletion can comprise a microdeletion. A deletion can comprise the deletion of a single base.

A genetic variation can be a genetic duplication. A duplication can be a mutation (e.g., a genetic aberration) in which a part of a chromosome or a sequence of DNA is copied and inserted back into the genome, A genetic duplication is any duplication of a region of DNA. A duplication can be a nucleic acid sequence that is repeated, often in tandem, within a genome or chromosome. A duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (e.g, repeated 1, 2, 3, 4, 5, 6, 7, 8, 9, or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases.

A genetic variation can be an insertion. An insertion can be the addition of one or more nucleotide base pairs into a nucleic acid sequence. An insertion is sometimes a microinsertion. An insertion comprises the addition of a segment of a chromosome into a genome, chromosome, or segment thereof, An insertion comprises the addition of an allele, a gene, an intron, an exon, any non-coding region, any coding region, segment thereof, or a combination thereof into a genome or segment thereof. An insertion comprises the addition (i.e., insertion) of the nucleic acid of unknown origin into a genome, chromosome, or segment thereof. An insertion comprises the addition (i.e., insertion) of a single base.

A genetic variation can be a single nucleotide polymorphism or single nucleotide variation. A single nucleotide polymorphism is a variation whereby a specific base within a genome is replaced with another base—e.g. A for C or G and this change is present throughout all cells of the organism including the germline and is thus inheritable. A single nucleotide polymorphism may be associated with an inherited disorder—such as a monogenic disease. Examples of monogenic diseases include but are not limited achondroplasia, adrenoleukodystrophy, alpha thalassaemia, alpha-1-antitrypsin deficiency, Alport syndrome, amyotrophic lateral sclerosis, beta thalassemia, Charcot-Marie-Tooth, congenital disorder of glycosylation type 1a, Crouzon syndrome, cystic fibrosis, Duchenne and Becker muscular dystrophy, dystonia 1. Torsion, Emery-Dreifuss muscular dystrophy, facioscapulohumeral dystrophy, familial adenomatous polyposis, familial amyloidotic polyneuropathy, familial dysautonomia, fanconi anaemia, Fragile X, glutaric aciduria type 1, haemophilia A and B, hemophagocytic lymphohistiocytosis, Holt-Oram syndrome, Huntington's disease, hyperinsulinernic hypoglycemia, hypokalaemic periodic paralysis, Incontinentia pigmenti, Lynch syndrome, Marfan syndrome, Menkes disease, metachromatic leukodystrophy, mucopolysaccharidosis type II (Hunter syndrome), multiple endocrine neoplasia (MEN2), multiple exostosis, myotonic dystrophy, neurofibromatosis type I and II, non-syndromic Sensorineural Deafness, Norrie syndrome, Osteogenesis imperfecta (brittle bone disease), polycystic kidney, autosomal dominant, polycystic kidney, autosomal recessive, Pompe's syndrome, sickle cell anaemia, Smith-Lemli-Opitz syndrome, spastic paraplegia 4, spinal and bulbar muscular atrophy, spinal muscular atrophy, spinocerebellar ataxia 1, 2 and 3, Spondylometaphyseal dysplasia. (Schmidt), Tay-Sachs disease, Treacher Collins, tuberous sclerosis, Von Hippel-Lindau syndrome, X-linked dystonia parkinsonism (XDP), X-linked agammaglobulinemia, leukemia, hereditary elliptocytosis and pyropoikilocytosis, autosomal recessive hypercholesterolemia, Fukuyama-type muscular dystrophy. Cystic fibrosis is a monogenic disease which may be caused in certain cases by a single nucleotide polymorphism. Single nucleotide variations are similar to single nucleotide polymorphisms but refer to nucleotide variations that occurs somatically, such as those that may occur in cancer and are not therefore inherited. The methods described herein are useful in determining the presence or absence of single nucleotide polymorphisms or variations in the nucleic acids of a sample.

Copy number variations, copy number alterations, copy number aberrations, aneuploidy, or copy number polymorphisms (collectively referred to as Copy Number Variants (CNVs)) are structurally variant regions in which copy number differences are observed between two or more genomes. Somatic CNVs have critical roles in the development of human cancers through the amplification of oncogenes and the deletion of tumor suppressors, Some CNVs have lethal or detrimental developmental effects on developing embryos, fetuses such as Down Syndrome caused by trisomy for Chromosome 21, Therefore, detecting CNVs from cfDNA and/or cfRNA may provide effective cancer and prenatal diagnosis and prognosis mechanism enabling treatment of cancer or the identification of embryos suitable for implantation as part of an in vitro fertilization procedure.

A copy number variation can be a fetal or embryo copy number variation. A copy number variation can be a maternal copy number variation. The subject methods described herein may be used to detect a copy number variation in the maternal, fetal, or embryo genome. For example, the copy number variation may be found in the genome of a pregnant female (e.g., a female subject bearing a fetus), the fetus carried by a pregnant female, a female subject that gave birth, a female capable of bearing a fetus, or an embryo fertilized in vitro for implantation in a female subject. A copy number variation can be a heterozygous copy number variation where the variation (e.g., duplication or deletion) is present on one allele of a genome. A copy number variation can be a homozygous copy number variation where the variation is present on both alleles of a genome. In some embodiments, a copy number variation is a heterozygous or homozygous fetal copy number variation. In some embodiments, a copy number variation is a heterozygous or homozygous maternal and/or fetal or embryo copy number variation. A copy number variation sometimes is present in a maternal genome and a fetal/embryo genome, a maternal genome and not a fetal/ embryo genome, or a fetal/embryo genome and not a maternal genome.

“Ploidy” refers to the number of chromosomes present in a cell, fetus/embryo or mother. In some embodiments, “Ploidy” is the same as “chromosome ploidy”. In humans, for example, autosomal chromosomes are typically present in pairs. For example, in the absence of a genetic variation, most humans have two of each autosomal chromosome (i.e., chromosomes 1-22). The presence of the normal complement of 2 autosomal chromosomes in a human is often referred to as euploid.

The methods described herein can be used to identify the presence or absence of one or more genetic variations that are associated with a medical condition or medical state. Non-limiting examples of medical conditions include those associated with intellectual disability (e.g., Down Syndrome), aberrant cell-proliferation (e.g., cancer), presence of a micro-organism nucleic acid (e.g., virus, bacterium, fungus, yeast), and preeclampsia.

### Fetal Gender

In some embodiments, the prediction of a fetal gender or gender-related disorder (e.g., sex chromosome aneuploidy) can be determined by the methods described herein. In some embodiments, a method in which fetal gender is determined can also comprise determining fetal fraction and/or the presence or absence of a fetal genetic variation (e.g., fetal chromosome aneuploidy).

Gender determination generally is based on a sex chromosome. In humans, there are two sex chromosomes, the X and Y chromosomes. The Y chromosome contains a gene, SRY, which triggers embryonic development as a male. The Y chromosomes of humans and other mammals also contain other genes needed for normal sperm production. Individuals with XX are female and XY are male and non-limiting variations, often referred to as sex chromosome aneuploidies, include X0, XYY, XXX and XXY. In some instances, males have two X chromosomes and one Y chromosome (XXY; Klinefelter's Syndrome), or one X chromosome and two Y chromosomes (XYY syndrome; Jacobs Syndrome), and some females have three X chromosomes (XXX; Triple X Syndrome) or a single X chromosome instead of two (X0; Turner Syndrome). In some instances, only a portion of cells in an individual is affected by a sex chromosome aneuploidy which may be referred to as mosaicism (e.g., Turner mosaicism). Other cases include those where SRY is damaged (leading to an XY female) or copied to the X (leading to an XX male).

In certain cases, it can be beneficial to determine the gender of a fetus in utero or of an embryo fertilized in vitro. For example, a patient (e.g., pregnant female) with a family history of one or more sex-linked disorders may wish to determine the gender of the fetus she is carrying to help assess the risk of the fetus inheriting such a disorder. Alternatively, a couple wishing to have a child by in vitro fertilization may wish to assess the risk that an implanted embryo may have inherited the sex-linked disorder. Sex-linked disorders include, without limitation, X-linked and Y-linked disorders. X-linked disorders include X-linked recessive and X-linked dominant disorders. Examples of X-linked recessive disorders include, without limitation, immune disorders (e.g., chronic granulomatous disease (CYBB), Wiskott-Aldrich syndrome, X-linked severe combined immunodeficiency, X-linked agammaglobulinemia, hyper-IgM syndrome type 1, IPEX, X-linked lymphoproliferative disease, Properdin deficiency), hematologic disorders (e.g., Hemophilia A, Hemophilia B, X-linked sideroblastic anemia), endocrine disorders (e.g., androgen insensitivity syndrome/Kennedy disease, KAL1 Kallmann syndrome, X-linked adrenal hypoplasia congenital), metabolic disorders (e.g., ornithine transcarbamylase deficiency, oculocerebrorenal syndrome, adrenoleukodystrophy, glucose-6-phosphate dehydrogenase deficiency, pyruvate dehydrogenase deficiency, Danon diseaseldlycogen storage disease Type IIb, Fabry's disease, Hunter syndrome, Lesch-Nyhan syndrome, Menkes disease/occipital horn syndrome), nervous system disorders (e.g., Coffin-Lowry syndrome, MASA syndrome, X-linked alpha thalassemia mental retardation syndrome, Siderius X-linked mental retardation syndrome, color blindness, ocular albinism, Norrie disease, choroideremia, Charcot-Marie-Tooth disease (CMIX2-3), Pelizaeus-Merzbacher disease, SMAX2), skin and related tissue disorders (e.g., dyskeratosis congenital, hypohidrotic ectodermal dysplasia (EDA), X-linked ichthyosis, X-linked endothelial corneal dystrophy), neuromuscular disorders (e.g., Beckers muscular dystrophy/Duchenne, centronuclear myopathy (MTM1), Conradi-Hünermann syndrome, Emery-Dreifuss muscular dystrophy 1), urologic disorders (e.g., Alport syndrome, Dent's disease, X-linked nephrogenic diabetes insipiclus), bone/tooth disorders (e.g., AMELX Amelogenesis imperfecta), and other disorders (e.g., Barth syndrome, McLeod syndrome, Smith-Fineman-Myers syndrome, Simpson-Golabi-Behmel syndrome, Mohr-Tranebjaerg syndrome, Nasodigitoacoustic syndrome). Examples of X-linked dominant disorders include, without limitation, X-linked hypophosphatemia, Focal dermal hypoplasia, Fragile X syndrome, Aicardi syndrome, Incontinentia pigmenti, Rett syndrome, CHILD syndrome, Lujan-Fryns syndrome, and Orofaciodigital syndrome 1. Examples of Y-linked disorders include, without limitation, male infertility, retinits pigmentosa, and azoospermia.

### Chromosome Abnormalities

The presence or absence of a fetal or embryo chromosome abnormality can be determined by using a method or composition described herein. Chromosome abnormalities include, without limitation, a gain or loss of an entire chromosome or a region of a chromosome comprising one or more genes. Chromosome abnormalities include monosomies, trisomies, polysomies, loss of heterozygosity, deletions and/or duplications of one or more nucleotide sequences (e.g., one or more genes), including deletions and duplications caused by unbalanced translocations. The terms “aneuploidy” and “aneuploid” as used herein refer to an abnormal number of chromosomes in cells of an organism. As different organisms have widely varying chromosome complements, the term “aneuploidy” does not refer to a particular number of chromosomes, but rather to the situation in which the chromosome content within a given cell or cells of an organism is abnormal. In some embodiments, the term “aneuploidy” herein refers to an imbalance of genetic material caused by a loss or gain of a whole chromosome, or part of a chromosome. An “aneuploidy” can refer to one or more deletions and/or insertions of a segment of a chromosome.

The term “monosomy” as used herein refers to when a particular chromosome or chromosomal segment is present only in a single copy. Partial monosomy can occur in unbalanced translocations or deletions, in which only a segment of the chromosome is present in a single copy. Monosomy of sex chromosomes (45, X) causes Turner syndrome, for example.

The term “disomy” refers to the presence of two copies of a chromosome. For organisms such as humans that have two copies of each chromosome (those that are diploid or “euploid”), disomy is the normal condition. For organisms that normally have three or more copies of each chromosome (those that are triploid or above), disomy is an aneuploid chromosome state. In uniparental disomy, both copies of a chromosome come from the same parent (with no contribution from the other parent). Angelman syndrome (AS) and Prader-Willi syndrome (PWS) are examples of disorders that can be caused by uniparental disomy. Detection of uniparental disomy using the methods described herein may therefore be useful in the determination of such disorders.

The term “euploid” in some embodiments, refers to a normal complement of chromosomes.

The term “trisomy” as used herein refers to the presence of three copies, instead of two copies, of a particular chromosome. The presence of an extra chromosome 21, which is found in human Down syndrome, is referred to as “Trisomy 21.” Trisomy 18 and Trisomy 13 are two other human autosomal trisomies. Trisomy of sex chromosomes can be seen in females (e.g., 47, XXX in Triple X Syndrome) or males (e.g., 47, XXY in Klinefelter's Syndrome; or 47, XYY in Jacobs Syndrome).

The terms “tetrasomy” and “pentasomy” as used herein refer to the presence of four or five copies of a chromosome, respectively. Although rarely seen with autosomes, sex chromosome tetrasomy and pentasomy have been reported in humans, including XXXX, XXXY, XXYY, XYYY, XXXXX, XXXXY, XXXYY, XXYYY, and XYYYY.

Chromosome abnormalities can be caused by a variety of mechanisms occurring in either mitosis or meiosis. Mechanisms include, but are not limited to (i) nondisjunction occurring as the result of a weakened mitotic or meiotic checkpoint, (ii) inactive mitotic checkpoints causing non-disjunction at multiple chromosomes, (iii) merotelic attachment occurring when one kinetochore is attached to both mitotic spindle poles, (iv) a multipolar spindle forming when more than two spindle poles form, (v) a monopolar spindle forming when only a single spindle pole forms, and (vi) a tetraploid intermediate occurring as a result of the monopolar spindle mechanism. Chromosomal abnormalities that occur as a result of meiotic errors are typically found throughout all cells of an embryo, whereas mitotic errors may be limited to one or a subset of cells of the embryo and thus result in mosaicism.

The terms “partial monosomy” and “partial trisomy” as used herein refer to an imbalance of genetic material caused by loss or gain of part of a chromosome. Partial monosomy or partial trisomy can result from an unbalanced translocation, where an individual carries a derivative chromosome formed through the breakage and fusion of two different chromosomes. In this situation, the individual would have three copies of part of one chromosome (two normal copies and the segment that exists on the derivative chromosome) and only one copy of part of the other chromosome involved in the derivative chromosome.

The term “mosaicism” as used herein refers to aneuploidy in some cells, but not all cells, of an organism. Certain chromosome abnormalities can exist as mosaic and non-mosaic chromosome abnormalities. For example, certain trisomy 21 individuals have mosaic Down syndrome and some have non-mosaic Down syndrome. As noted above, different mechanisms can lead to mosaicism. For example, (i) an initial zygote may have three copies of chromosome 21, which normally would result in simple trisomy 21, but during cell division, one or more of the cells lose one of the copies of chromosome 21; and (ii) an initial zygote may have two copies of chromosome 21, but during the course of cell division, one of the copies of chromosome 21 were duplicated. Somatic mosaicism likely occurs through mechanisms distinct from those typically associated with genetic syndromes involving complete or mosaic aneuploidy. Somatic mosaicism has been identified in certain types of cancers and neurons, for example. In certain instances, trisomy 12 has been identified in chronic lymphocytic leukemia (CLL) and trisomy 8 has been identified in acute myeloid leukemia, (AML). Also, genetic syndromes in which an individual is predisposed to breakage of chromosomes (chromosome instability syndromes) are frequently associated with increased risk for various types of cancer, thus highlighting the role of somatic aneuploidy in carcinogenesis. Methods and protocols described herein can identify the presence or absence of non mosaic and mosaic chromosome abnormalities.

### Preeclampsia

In some embodiments, the presence or absence of preeclampsia is determined by using a method or apparatus described herein. Preeclampsia is a condition in which hypertension arises in pregnancy (i.e., pregnancy-induced hypertension) and is associated with significant amounts of protein in the urine. In some instances, preeclampsia also is associated with elevated levels of extracellular nucleic acid and/or alterations in methylation patterns. For example, a positive correlation between extracellular fetal-derived hypermethylated RASSF1A levels and the severity of pre-eclampsia has been observed. In certain examples, increased DNA methylation is observed for the H19 gene in preeclamptic placentas compared to normal controls.

Preeclampsia is one of the leading causes of maternal and fetal/neonatal mortality and morbidity worldwide. Circulating cell-free nucleic acids in plasma and serum are novel biomarkers with promising clinical applications in different medical fields, including prenatal diagnosis, Quantitative changes of cell-free fetal (cff)DNA in maternal plasma as an indicator for impending preeclampsia have been reported in different studies, for example, using real-time quantitative PCR for the male-specific SRY or DYS 14 loci. In cases of early-onset preeclampsia, elevated levels may be seen in the first trimester. The increased levels of cffDNA before the onset of symptoms may be due to hypoxia/reoxygenation within the intervillous space leading to tissue oxidative stress and increased placental apoptosis and necrosis. In addition to the evidence for increased shedding of cffDNA into the maternal circulation, there is also evidence for reduced renal clearance of cffDNA in preeclampsia. As the amount of fetal DNA is currently determined by quantifying Y-chromosome-specific sequences, alternative approaches such as measurement of total cell-free DNA or the use of gender-independent fetal epigenetic markers, such as DNA methylation, offer an alternative. Cell-free RNA of placental origin is another alternative biomarker that may be used for screening and diagnosing preeclampsia in clinical practice. Fetal RNA is associated with subcellular placental particles that protect it from degradation. Fetal RNA levels sometimes are ten-fold higher in pregnant females with preeclampsia compared to controls, and therefore is an alternative biomarker that may be used for screening and diagnosing preeclampsia in clinical practice.

### Pathogens

In some embodiments, the presence or absence of a pathogenic condition is determined by a method or composition described herein. A pathogenic condition can be caused by infection of a host by a pathogen including, but not limited to, a bacterium, virus, or fungus. Since pathogens typically possess nucleic acid (e.g., genomic DNA, genomic RNA, mRNA) that can be distinguishable from host nucleic acid, methods and apparatus provided herein can be used to determine the presence or absence of a pathogen. Often, pathogens possess nucleic acid with characteristics unique to a particular pathogen such as, for example, epigenetic state and/or one or more sequence variations, duplications, and/or deletions. Thus, methods provided herein may be used to identify a particular pathogen or pathogen variant (e.g., strain).

### Cancers

In some embodiments, the presence or absence of a cell proliferation disorder (e.g., cancer) is determined by using a method or composition described herein. For example, levels of cell-free nucleic acid in serum can be elevated in patients with various types of cancer compared with healthy patients. Patients with metastatic diseases, for example, can sometimes have serum DNA levels approximately twice as high as non-metastatic patients. Patients with metastatic diseases may also be identified by cancer-specific markers and/or certain single nucleotide polymorphisms or short tandem repeats, for example. Non-limiting examples of cancer types that may be positively correlated with elevated levels of circulating DNA include breast cancer, colorectal cancer, gastrointestinal cancer, hepatocellular cancer, lung cancer, melanoma, non-Hodgkin lymphoma, leukemia, multiple myeloma, bladder cancer, hepatoma, cervical cancer, esophageal cancer, pancreatic cancer, and prostate cancer. Various cancers can possess, and can sometimes release into the bloodstream, nucleic acids with characteristics that are distinguishable from nucleic acids from non-cancerous healthy cells, such as, for example, epigenetic state and/or sequence variations, duplications, and/or deletions. Such characteristics can, for example, be specific to a particular type of cancer. Thus, it is further contemplated that a method provided herein can be used to identify a particular type of cancer.

### Sequencing for Genetic Analysis

In some embodiments, nucleic acids (e.g., nucleic acid fragments, sample nucleic acid, cell-free nucleic acid) may be sequenced. In some embodiments, a full or substantially full sequence is obtained and sometimes a partial sequence is obtained. In some embodiments, a nucleic acid is not sequenced, and the sequence of nucleic acid is not determined by a sequencing method, when performing a method described herein. Sequencing, mapping and related analytical methods are known in the art and only certain aspects of such processes are described hereafter.

As used herein, “reads” (i.e., “a read”, “a sequence read”) are nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads).

In some embodiments, the nominal, average, mean, or absolute length of single-end reads sometimes is about 20 contiguous nucleotides to about 300 contiguous nucleotides, sometimes about 30 contiguous nucleotides to about 200 contiguous nucleotides, and sometimes about 40 contiguous nucleotides or about 100 contiguous nucleotides. In some embodiments, the nominal, average, mean, or absolute length of single-end reads is about 50 to about 80 bases in length.

In certain embodiments, the nominal, average, mean or absolute length of the paired-end reads sometimes is about 10 contiguous nucleotides to about 300 contiguous nucleotides, sometimes about 30 contiguous nucleotides to about 200 contiguous nucleotides, and sometimes about 40 contiguous nucleotides or about 100 contiguous nucleotictes. In some embodiments, the nominal, average, mean, or absolute length of single-end reads is about 50 to about 80 bases in length.

In some instances, long read sequencing technologies, such as those provided by PacBio and Oxford Nanopore, are employed in practicing embodiments of the invention. In the case of long reads, reads may vary, and in some instances be 1 kb to 40 kb in length, such as 1 kb to 5 kb in length.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample of interest, e.g., the blood of a pregnant female, can be reads from a mixture of fetal and maternal nucleic acid. Similarly, sequence reads obtained from sequencing libraries made from spent media or culture media used for culturing in vitro fertilized embryos using the methods described herein can be reads from a mixture of the embryo and maternal nucleic acid.

Sequence reads can be mapped and the number of reads or sequence tags mapping to a specified nucleic acid region (e.g., a chromosome, a bin, a genomic section) are referred to as counts. The term bin is used to refer to a contiguous length of genomic sequence within a chromosome. Bins are assigned arbitrarily—such as every 1 million bases, such as every 2 million bases, every 100 kilo bases, etc. Bin may also be assigned variable length based on factors such as GC content or presence of repeats or mappable sequence content, etc. Assigning reads to bins is useful for determining copy number variation according to the methods disclosed in WO2017083310. Briefly, bins can be divided into 1 MB intervals for mapping the reads to the bins. A subset of bins, such as 400-800 bins, can be selected for normalizing all the bins for a sample. The normalized reads or effective counts can further be used for determining Calculated Copy Number (CCN) by dividing the effective counts for each bin in each sample by the median count value for the corresponding bin in a reference dataset. In some cases, the median count value in the reference dataset can be the median count value across all reference samples, for each bin. A significant deviation, e.g., as determined using any appropriate statistical test known in the art, between the median values from the reference dataset and the CCN values from the test sample may indicate a copy number variation in the test sample relative to the reference. In some embodiments, the count can be determined not simply based on the expression level of the gene or read, but by reference to specific mutations, SNPs, present in the sample. By determining the relative ratio of the wild type allele and a mutant allele at a given locus where the sample is heterozygous for the mutation, either by PCR or sequencing, it is also possible to determine the frequency or copy number of each allele. Use of allele frequency or copy number may be useful in distinguishing fetal from maternal nucleic acid for use in the methods of the invention.

In some embodiments, counts can be manipulated or transformed (e.g., normalized, combined, added, filtered, selected, averaged, derived as a mean, the like, or a combination thereof). In some embodiments, counts can be transformed to produce normalized counts. Normalized counts for multiple genomic sections or bins can be provided in a profile (e.g., a genomic profile, a chromosome profile, a profile of a segment of a chromosome).

In some embodiments, one nucleic acid sample from one individual is sequenced. In certain embodiments, nucleic acid samples from two or more biological samples, where each biological sample is from one individual or two or more individuals, are pooled and the pool is sequenced. In the latter embodiments, a nucleic acid sample from each biological sample often is identified by one or more unique identification tags.

In some embodiments, a fraction of the genome is sequenced, which sometimes is expressed in the amount of the genome covered by the determined nucleotide sequences (e.g., “fold” coverage less than 1). When a genome is sequenced with about 1-fold coverage, roughly 100% of the nucleotide sequence of the genome is represented by reads. A genome also can be sequenced with redundancy, where a given region of the genome can be covered by two or more reads or overlapping reads (e.g., “fold” coverage greater than 1). In some embodiments, a genome is sequenced with about 0.01-fold to about 100-fold coverage, about 0.2-fold to 20-fold coverage, or about 0.2-fold to about 1-fold coverage (e.g., about 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage).

In certain embodiments, a subset of nucleic acid fragments is selected before sequencing. In certain embodiments, hybridization-based techniques (e.g., using oligonucleotide arrays or hybrid capture using oligonucleotides associated with beads to enable capture and “pull-down” of the fragments of interest, such as Agilent's SureSelect technology—https://www.agilent.com/en/product/next-generation-sequencing/hybridization-based-next-generation-sequencing-ngs/dna-seq-reagents-kits-library-preparation-kits/sureselectxt-reagent-kits-232859)) can be used to the first select for nucleic acid sequences from certain chromosomes (e.g., a potentially aneuploid chromosome and other chromosome(s) not involved in the aneuploidy tested). In some embodiments, the nucleic acid can be fractionated by size (e.g., by gel electrophoresis, size exclusion chromatography, or by microfluidics-based approach). For example, in certain instances, the cell-free nucleic acid from spent media of the in vitro fertilized embryos can be enriched by selecting for nucleic acid having a lower molecular weight (e.g., less than 300 base pairs, less than 200 base pairs, less than 150 base pairs, less than 100 base pairs).

In some embodiments, a portion or subset of a pre-selected set of nucleic acid fragments is sequenced randomly. In some embodiments, the nucleic acid is amplified before sequencing. In some embodiments, a portion or subset of the nucleic acid is amplified before sequencing.

In some embodiments, a sequencing library is prepared before or during a sequencing process. Methods for preparing a sequencing library are known in the art and commercially available platforms may be used for certain applications. Certain commercially available library platforms may be compatible with certain nucleotide sequencing processes described herein. For example, one or more commercially available library platforms may be compatible with a sequencing by synthesis process. In some embodiments, a ligation-based library preparation method is used (e.g., ILLUMINA TRUSEQ, Illumina, San Diego Calif.). Ligation-based library preparation methods typically incorporate an index sequence at the initial ligation step and often can be used to prepare samples for single-read sequencing, paired-end sequencing, and multiplexed sequencing. In some embodiments, a transposon-based library preparation method is used (e.g., EPICENTRE NEXTERA, Epicentre, Madison Wis.). Transposon-based methods typically use in vitro transposition to simultaneously fragment and tag DNA in a single-tube reaction (often allowing incorporation of platform-specific tags and optional barcodes) and prepare sequencer-ready libraries.

Any sequencing method suitable for conducting methods described herein can be utilized.

In some embodiments, a high-throughput sequencing method is used. High-throughput sequencing methods generally involve clonally amplified DNA templates or single DNA molecules that are sequenced in a massively parallel fashion within a flow cell (e.g., as described in Metzker M Nature Rev 11:31-46 (2010); Volkerding et al. Clin Chem 55:641-658 (2009)). Such sequencing methods also can provide digital quantitative information, where each sequence read is a countable “sequence tag” or “count” representing an individual clonal DNA template, a single DNA molecule, bin or chromosome. Next-generation sequencing techniques capable of sequencing DNA in a massively parallel fashion are collectively referred to herein as “massively parallel sequencing” (MPS). High-throughput sequencing technologies include, for example, sequencing-by-synthesis with reversible dye terminators, sequencing by oligonucleotide probe ligation, pyrosequencing, and real-time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single-molecule sequencing, single-molecule real-time (SMRT) sequencing, nanopore sequencing, ION Torrent and RNA polymerase (RNAP) sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In certain sequencing by synthesis procedures, for example, template DNA (e.g., circulating cell-free DNA (ccfDNA)) sometimes can be fragmented into lengths of several hundred base pairs in preparation for library generation. In some embodiments, library preparation can be performed without further fragmentation or size selection of the template DNA (e.g., ccfDNA). Sample isolation and library generation may be performed using automated methods and apparatus, in certain embodiments.

In certain sequencing by synthesis procedures, for example, adapter oligonucleotides are complementary to the flow-cell anchors, and sometimes are utilized to associate the modified template DNA (e.g., end-repaired and single nucleotide extended) with solid support, such as the inside surface of a flow cell, for example. In some embodiments, the adapter also includes identifiers (i.e., indexing nucleotides, or “barcode” nucleotides (e.g., a unique sequence of nucleotides usable as an identifier to allow unambiguous identification of a sample and/or chromosome)), one or more sequencing primer hybridization sites (e.g., sequences complementary to universal sequencing primers, single-end sequencing primers, paired-end sequencing primers, multiplexed sequencing primers, and the like), or combinations thereof (e.g., adapter/sequencing, adapter/identifier, adapter/identifier/sequencing). Identifiers or nucleotides contained in an adapter often are six or more nucleotides in length, and frequently are positioned in the adaptor such that the identifier nucleotides are the first nucleotides sequenced during the sequencing reaction. In certain embodiments, identifier nucleotides are associated with a sample but are sequenced in a separate sequencing reaction to avoid compromising the quality of sequence reads. Subsequently, the reads from the identifier sequencing and the DNA template sequencing are linked together and the reads de-multiplexed. After linking and de-multiplexing, the sequence reads and/or identifiers can be further adjusted or processed as described herein.

In certain sequencing by synthesis procedures, the utilization of identifiers allows multiplexing of sequence reactions in a flow cell lane, thereby allowing the analysis of multiple samples per flow cell lane. The number of samples that can be analyzed in a given flow cell lane often is dependent on the number of unique identifiers utilized during library preparation and/or probe design. A method described herein can be performed using any number of unique identifiers (e.g., 4, 8, 12, 24, 48, 96, or more). The greater the number of unique identifiers, the greater the number of samples and/or chromosomes, for example, that can be multiplexed in a single flow cell lane. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell.

Other sequencing methods that may be used to conduct methods herein include digital PCR, for example digital droplet PCR, and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g., fetal/embryo alleles and maternal alleles). Alleles can be enumerated to determine copy number. In sequencing by hybridization, the method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization of the beads can be identified and used to identify the plurality of polynucleotide sequences within the sample.

In some embodiments, chromosome-specific sequencing is performed. In some embodiments, chromosome-specific sequencing is performed utilizing DANSR (digital analysis of selected regions). Digital analysis of selected regions enables simultaneous quantification of hundreds of loci by cfDNA-dependent catenation of two locus-specific oligonucleotides via an it ‘bridge’ oligo to form a PCR template. In some embodiments, chromosome-specific sequencing is performed by generating a library enriched in chromosome-specific sequences, in some embodiments, sequence reads are obtained only for a selected set of chromosomes. In some embodiments, sequence reads are obtained only for chromosomes 21, 18, and 13.

### Mapping Reads

Mapping nucleotide sequence reads (i.e., sequence information from a fragment whose physical genomic position is unknown) can be performed in several ways, and often comprises alignment of the obtained sequence reads with a matching sequence in a reference genome (e.g., Li et al., “Mapping short DNA sequencing reads and calling variants using mapping quality score,” Genome Res., 2008 Aug. 19.) In such alignments, sequence reads generally are aligned to a reference sequence and those that align are designated as being “mapped” or a “sequence tag.” In some embodiments, a mapped sequence read is referred to as a “hit” or a “count”. In some embodiments, mapped sequence reads are grouped together according to various parameters and assigned to particular genomic sections, which are discussed in further detail below.

As used herein, the terms “aligned”, “alignment”, or “aligning” refer to two or more nucleic acid sequences that can be identified as a match (e.g., 100% identity) or partial match. Alignments can be done manually or by a computer algorithm, examples including the Efficient Local Alignment of Nucleotide Data (ELAND) computer program distributed as part of the Illumina Genomics Analysis pipeline. The alignment of a sequence read can be a 100% sequence match.

In some embodiments, alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4, or 5 mismatches. Two or more sequences can be aligned using either strand. In some embodiments, a nucleic acid sequence is aligned with the reverse complement of another nucleic acid sequence.

Various computational methods can be used to map each sequence read to a genomic section. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with sequences in a reference genome. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate genomic sections (described hereafter), for example.

The term “sequence tag” is herein used interchangeably with the term “mapped sequence tag” to refer to a sequence read that has been specifically assigned i.e., mapped, to a larger sequence e.g., a reference genome, by alignment. Mapped sequence tags are uniquely mapped to a reference genome i.e., they are assigned to a single location to the reference genome. Tags that can be mapped to more than one location on a reference genome i.e., tags that do not map uniquely are not included in the analysis. A “sequence tag” can be a nucleic acid (e.g., DNA) sequence (i.e., read) assigned specifically to a particular genomic section and/or chromosome (i.e., one of chromosomes 1-22, X or Y for a human subject). A sequence tag may be repetitive or non-repetitive within a single segment of the reference genome (e.g., a chromosome). In some embodiments, repetitive sequence tags are eliminated from further analysis (e.g., quantification).

In some embodiments, a read may uniquely or non-uniquely map to sections in the reference genome. A read is considered to be “uniquely mapped” if it aligns with a single sequence in the reference genome. A read is considered to be “non-uniquely mapped” if it aligns with two or more sequences in the reference genome. In some embodiments, non-uniquely mapped reads are eliminated from further analysis (e.g., quantification). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read to be mapped to a reference sequence.

As used herein, the term “reference genome” can refer to any particular known, sequenced, or characterized genome, whether partial or complete, of any organism or virus which may be used to reference identified sequences from a subject. For example, a reference genome used for human subjects as well as many other organisms can be found at the National Center for Biotechnology Information at the world wide web universal source code address ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, where a sample nucleic acid is from a pregnant female, a reference sequence sometimes is not from the fetus, the mother of the fetus, or the father of the fetus, and is referred to herein as an “external reference.” A maternal reference may be prepared and used in some embodiments. A reference sometimes is prepared from maternal nucleic acid (e.g., cellular nucleic acid). When a reference from the pregnant female is prepared (“maternal reference sequence”) based on an external reference, reads from DNA of the pregnant female that contains substantially no fetal DNA often are mapped to the external reference sequence and assembled. In certain embodiments, the external reference is from the DNA of an individual having substantially the same ethnicity as the pregnant female. A maternal reference sequence may not completely cover the maternal genomic DNA (e.g., it may cover about 50%, 60%, 70%, 80%, 90%, or more of the maternal genomic DNA), and the maternal reference may not perfectly match the maternal genomic DNA sequence (e.g., the maternal reference sequence may include multiple mismatches).

In some embodiments, a reference for copy number calling can be prepared by using collections of normal or euploid cell lines, such as PBMCs or normal embryo trophectoderm cells.

In some other embodiments, a reference for copy number calling can be prepared by using collections of cell culture media in which normal or euploid cell lines were cultured. In some embodiments, a reference for copy number calling can be prepared by using collections of normal or euploid cell lines and cell culture media in which normal or euploid embryos were cultured.

In some embodiments, mappability is assessed for a genomic region (e.g., genomic section, genomic portion, bin). Mappability is the ability to unambiguously align a nucleotide sequence read to a section of a reference genome, typically up to a specified number of mismatches, including, for example, 0, 1, 2, or more mismatches. For a given genomic region, the expected mappability can be estimated using a sliding-window approach of a preset read length and averaging the resulting read-level mappability values. Genomic regions comprising stretches of unique nucleotide sequences sometimes have a high mappability value.

In some embodiments, a mapping feature is assessed for a genomic region (e.g., genomic section, genomic portion, bin). Mapping features can include any feature of a genomic region that can directly or indirectly influence the mapping of sequence reads thereto. Mapping features can include, for example, a measure of mappability, nucleotide sequence, nucleotide composition, location within the genome, location within a chromosome, proximity to certain regions within a chromosome, and the like. In some embodiments, a mapping feature can be a measure of mappability for the genomic region. In some embodiments, a mapping feature can be GC content of the genomic region. In some embodiments, a mapping feature can influence experimental bias (e.g., mappability bias, GC bias) for certain genomic regions, as described in further detail herein.

In some embodiments, the use of one or more reference samples known to be free of a genetic variation in question can be used to generate a reference profile, which may result in a predetermined value representative of the absence of the genetic variation, and often deviates from a predetermined value in areas corresponding to the genomic location in which the genetic variation is located in the test subject if the test subject possessed the genetic variation or is suspected of possessing the variation. In some cases, the test subject can be a sample collected from the subject (e.g., blood) or a sample derived from the subject (e.g., culture media). In test subjects at risk for or suffering from a medical condition associated with a genetic variation, the numerical value for the selected genomic section or sections is expected to vary significantly from the predetermined value for non-affected genomic locations. In certain embodiments, the use of one or more reference samples known to carry the genetic variation in question can be used to generate a reference median count profile, which may result in a predetermined value representative of the presence of the genetic variation, and often deviates from a predetermined value in areas corresponding to the genomic location in which a test subject does not carry the genetic variation. In test subjects not at risk for or suffering from a medical condition associated with a genetic variation, the numerical value for the selected genomic section or sections is expected to vary significantly from the predetermined value for affected genomic locations.

### Kits

Aspects of the present disclosure also include kits. The kits may include, e.g., buffers, such as lysis buffer, washing buffer, and elution buffer for the isolation of nucleic acids. The kits may include the functionalized beads or particles, such as magnetic beads. The kits may also include at least one reagent for the nucleic acid assay(s). Further, the kits may include tubes, multi-well plates, columns, etc.

In addition to the above-mentioned components, a subject kit may further include instructions for using the components of the kit, e.g., to practice the subject methods as described above. The instructions are generally recorded on a suitable recording medium. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, CD-ROM, diskette, Hard Disk Drive (HDD) etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The following example(s) is/are offered by way of illustration and not by way of limitation.

### EXAMPLES

### Example 1: Optimization of a Single-Tube Workflow

A single-tube workflow was optimized by combining the DNA purification and whole genome amplification (WGA) procedures into a single tube. The DNA purification procedure was performed by using the NucleoMag® cfDNA Kit (Takara Bio USA, Mountain View, CA) following the manufacturer's instructions with some modifications as described below. The WGA procedure was performed using the PicoPLEX V1 WGA method (Takara Bio USA, Mountain View, CA) following the manufacturer's instructions. The NucleoMag® cfDNA Kit first isolates the DNA from a sample by lysing the sample and then purifying the DNA by using magnetic beads. The DNA isolation step was carried out by adding 2 μL of Proteinase K followed by the addition of 36 μL of Lysis Buffer MCF1 to each tube containing the sample. The sample input volume varied between 10 and 40 μL. The contents of the tubes were briefly mixed by vortexing followed by spinning the tube contents to collect the contents to the bottom of the tube by using a centrifuge and incubating the tubes at room temperature for 15 min.

NucleoMag® P-Beads, magnetic beads, were warmed up to room temperature for at least 30 min, vortexed until they appeared homogenous in the tube, and quickly spun down before use. A mastermix containing 1 μL of NucleoMag® P-Beads and 119 μL of Binding Buffer MCF2 was added to each sample tube. The sample tubes were mixed briefly by vortexing and incubated for 10 min at room temperature. After incubation, tube contents were briefly spun to collect the contents to the bottom of the tube by using a centrifuge and placed on a magnetic separator for 2 min or until beads were pelleted on the side of the tube and the supernatant was clear. Once the beads were pelleted, the supernatant was discarded by being careful not to touch the pellet with the pipette tip. The tubes were removed from the magnetic separator and the pelleted beads were washed twice. The beads were first washed with Wash Buffer MCF3 and then with Wash Buffer MCF4 by adding 100 μL of wash buffer to each sample. The tubes were then vortexed briefly to resuspend the pellet in wash buffer and incubated for 2 min at room temperature. After incubation, the tube contents were collected to the bottom of the tube by briefly spinning the contents down using a centrifuge and placed on a magnetic separator for 2 min or until the beads were pelleted on the side of the tube and the supernatant was clear. Once the beads were pelleted, the supernatant was discarded by being careful not to touch the pellet with the pipette tip. The tubes were briefly spun to collect the liquid from the side of the tube or plate well and then placed on the magnetic separation device for 30 sec to remove any residual buffer with a fine pipette tip. The magnetic beads were air-dried at room temperature for a maximum of 2 min or until the pellet was no longer shiny, but before cracks appeared.

The tubes were removed from the magnetic separator and cfDNA from the magnetic beads, P-beads, was eluted directly into the WGA reaction mix from Takara Bio USA PicoPLEX WGA V1 (R30050) (Takara Bio USA, Mountain View, CA) by pipetting in a mastermix containing 29.84 WGA Buffer, 1 μL WGA enzyme, and 44.24 nuclease-free water (754 total) into each tube. The beads were vortexed gently at medium speed or by pipetting the pellet up and down ten times. The tubes were spun briefly to collect the contents at the bottom of the tube followed by placing them into a thermocycler with the heated lid set to 100° C.-105° C. A Whole Genome Amplification Reaction was performed using the following cycling conditions:

| Number of cycles | Temperature | Duration | |
|---|---|---|---|
| 1 | 95° C. | 2 | min |
| 12 | 95° C. | 15 | sec |
| | 15° C. | 50 | sec |
| | 25° C. | 40 | sec |
| | 35° C. | 30 | sec |
| | 65° C. | 40 | sec |
| | 75° C. | 40 | sec |
| 14 | 95° C. | 15 | sec |
| | 65° C. | 1 | min |
| | 75° C. | 1 | min |
| 1 | 75° C. | 5 | min |

| | |
|---|---|
| 4° C. | Hold |

At the end of amplification, the tubes were spun briefly to collect the contents at the bottom of the tube and the samples were either stored in the thermal cycler at 4° C. overnight or transferred to −20° C. for long term storage.

NGS libraries were prepared from the WGA product using the Smart-Seq Library Preparation Kit (Catalog no. R400746; Takara Bio USA, Mountain View, CA) and Unique Dual Index Kit (Catalog no. R400744; Takara Bio USA, Mountain View, CA)), Takara Bio USA, Mountain View, CA, following the manufacturer protocol. Libraries were pooled in equimolar concentration, sequenced in a single run, and demultiplexed using the UDI sequence to identify which reads originate from each sample.

Sequenced reads were demultiplexed, trimmed, and then aligned to the human genome using a genome mapping algorithm followed by GC bias correction. Read depth was calculated for 1MB bins across the human genome. Bins were filtered and normalized using custom Takara Bio USA algorithms resulting in an effective count of reads per bin. The calculated copy number (CCN) for each bin was calculated using a reference set derived from a dataset made of euploid male and female samples derived from the same workflow. CCN values were then inputted into Takara Bio USA custom smoothing protocols. Genomic regions with abnormal copy number were identified using a custom Takara Bio USA CCN abnormality calling algorithm.

### Example 2: Single-Tube Workflow Using Cell-Free DNA

Cell-free DNA isolated from the culture media of human embryonic cell line SA002 was used to assess the performance of the optimized single-tube workflow which combines the DNA isolation and PicoPLEX WGA procedures in a single tube. A sample containing 0.2 pg/μL of cfDNA was used to compare the standard workflow (no beads) vs the single-tube workflow (P-beads) disclosed herein at input volumes of 5 μL and 30 μL, respectively. The new workflow resulted in increased WGA yield and decreased the average WGA product size (FIG. 4). This new method was tested and proven to be compatible with cfDNA diluted in several standard human embryo culture media: G-2 PLUS media (Vitrolife), Global+Quinn's Advantage Serum Substitute, Sage 1-Step (Vitrolife), Geri Medium (Geri) and Continuous Single Culture Complete with HSA media (CSCM) (Irvine Scientific). (FIG. 5, panel A, FIG. 5, panel B). Additionally, this assay was tested with increasing concentrations of serum protein substitute (SPS). The assay was compatible with all concentrations of SPS tested, up to 15 mg/ml (FIG. 5, panel C, FIG. 5, panel D).

WGA products derived from this new workflow yielded more DNA input for enzymatic fragmentation-based library preparation chemistries than the standard protocol. One ng of WGA product from the standard no bead vs P-bead WGA protocols were input into the Smart-Seq

Library Preparation Kit. The WGA product produced by the bead-based WGA chemistry yielded more library product compared to the standard no bead WGA chemistry (FIG. 6).

These libraries were sequenced on an Illumina MiSeq, downsampled to 1 million reads, and analyzed for their ability to detect changes in calculated copy number (CCN) through a custom copy number variation analysis software. The cell-free DNA derived from a previously tested human embryonic stem cell line that was verified to be female by identifying two copies of the X chromosome and with the trisomy on chromosome 13. The sequencing data from both the original WGA chemistry (FIG. 7, panel A) and the single-tube workflow comprising bead purification and WGA (FIG. 7, panel B) detected the trisomy on chromosome 13 and two copies of the X chromosome. The sequencing data from the libraries prepared from the optimized single-tube workflow had a higher mapping rate to the human genome (FIG. 8, panel A). The sequencing data from the libraries prepared from the single-tube workflow also had lower noise in the CCN plot, measured by the derivative log ratio score (DLRS) of the 1 MB CCN values (FIG. 8, panel B).

### Example 3: Magnetic Beads Compatibility for cfDNA Purification into Single Tube PicoPLEX WGA Workflow

Additional bead products were tested and compared to P-beads for compatibility as part of the novel single tube sample preparation method for PicoPLEX WGA. The additional bead products tested were AMPure XP beads (Beckman Coulter), Apostle MiniMax™ High Efficiency Isolation Kit (Apostle), Mag-Bind® TotalPure NGS (Omega Bio-Tek) and the Mag-Bind® cfDNA Kit (Omega Bio-Tek). Similar to the P-bead workflow described in Example 1, additional bead products were tested for cfDNA capture following the lysis step followed by direct elution into the

WGA mastermix and amplification with beads in the tube. Each bead product had unique binding buffers and wash buffers compared to the P-bead method described in Example 1 as well as between bead products. In all cases, the binding buffer tested was the one included with the bead product. For the Apostle MiniMax High Efficiency Isolation Kit and the Mag-Bind cfDNA Kit, the wash buffer was included in the original product. For the remaining bead products tested, an ethanol-based wash buffer was tested. All bead products tested were compatible with the new sample preparation method into PicoPLEX WGA chemistry producing WGA products with a similar yield as P-beads (FIG. 9).

### Example 4: Non-Invasive Preimplantation Genetic Testing for Aneuploidies (niPGT-A) Using the Single-Tube Workflow

Embryo spent media samples, collected from human embryos cultured for in vitro fertilization, were assessed for copy number variation by niPGT-A using the single-tube workflow which combines the bead purification and WGA procedures in a single tube. The new workflow successfully amplified DNA from all spent media samples tested. Spent media samples containing greater amounts of cfDNA resulted in larger WGA yields (FIG. 10, panel A) and larger WGA product average size (FIG. 10, panel B). Libraries were prepared from equal amounts of WGA products using the Takara Bio USA Smart-Seq Library Preparation Kit and the Unique Dual Index Kit. Library yield scaled with the original cfDNA concentration in the spent media sample (FIG. 10, panel C), while library size was consistent across all samples (FIG. 10, panel D).

Smart-Seq libraries were sequenced and analyzed using a custom Takara Bio USA Analysis protocol. CCN noise, quantified using the derivative log ratio score (DLRS), showed that CCN noise decreased with increasing spent media cfDNA input into the chemistry (FIG. 11). Chromosomal abnormalities identified using this niPGT-A workflow and analysis were compared to PGT-A results from TE biopsy samples taken from the accompanying embryo for each spent media culture. Two examples of such comparison are provided in FIGS. 12 and 13. PGT-A analysis on the TE biopsy was performed using either the VeriSeq PGS kit and analyzed on BlueFuse Multi Analysis Software (FIG. 12, panel C and FIG. 12, panel D) following the manufacturer's instructions or using Veriseq and an alternative Software tool (FIG. 13 panel C and FIG. 13 panel D). CNV plots generated using the Takara Bio USA Analysis software and using the novel single-tube workflow (FIG. 12 panels A and B and FIG. 13 panels A and B) had a very similar CCN noise profile to CNV plots generated by BlueFuse Multi Analysis Software using VeriSeq chemistry (FIG. 12) or the alternative software tool (FIG. 13).

CNV concordance was observed between the two CNV plots for euploid (compare FIG. 12, panel A and FIG. 12, panel C) and aneuploid (compare FIG. 12, panel B and FIG. 12, panel D) samples from one IVF center. Similar results were obtained using spent media samples from another IVF center (FIG. 13). Again, CNV concordance was observed between the two CNV plots for euploid (compare FIG. 13, panel A and FIG. 13, panel C) and aneuploid (compare FIG. 13, panel B and FIG. 13, panel D). The overall clinical concordance across all samples and IVF clinics was 73%.

### Example 5: Detection of Mosaicism Using the Single-Tube Workflow and Custom Software

The ability to detect mosaic signals from cfDNA was tested by mixing two characterized Coriell genomic DNA samples at 10% intervals. These Coriell gDNA samples were NA09367: 46 XX, +6 35.2MB, and NA11672: 46 XY, -10: 26.2MB. Genomic DNA was first mechanically sheared to an average of 550 bp to mimic cfDNA fragments. Four picograms of sheared gDNA were input into WGA plus bead purification. Two nanograms of WGA product was used as an input into the Smart-Seq Library Preparation Kit. Libraries were sequenced on an Illumina MiSeq and reads were downsampled to 1 million per sample. CNV analysis of mosaic samples sequenced reads was performed using a custom Takara Bio USA Analysis Software, which detected mosaic signals on both the autosomes and sex chromosomes according to the input mixture of gDNA samples (FIG. 14).

### Example 6: Qualitative and Quantitative Assessment of DNA

DNA, for example, cell-free DNA, was assessed qualitatively to determine the fragmentation or integrity of DNA and quantitively to determine the concentration of DNA in a sample. For assessing the DNA, two primer pairs were designed to amplify DNA from a highly repetitive genomic element from the human genome. One primer pair was designed to amplify a short fragment of DNA, small enough to be protected by a single nucleosome, i.e., approximately 180 base pairs. The second primer pair was designed to amplify a larger fragment of DNA, yet still small enough to be protected by two nucleosomes, i.e., approximately 360 base pairs

Primer sequences for amplifying the short fragment:

Forward primer:
(SEQ ID NO: 01)
5′ CCTGAGGTCAGGAGTTCGAG 3′
Reverse primer:
(SEQ ID NO: 02)
5′ CCCGAGTAGCTGGGATTACA 3′

Primer sequences for amplifying the long fragment:

Forward primer:
(SEQ ID NO: 03)
5′ GTGGCTCACGCCTGTAATC 3′
Reverse primer:
(SEQ ID NO: 04)
5′ CAGGCTGGAGTGCAGTGG 3′

Each primer pair was amplified by quantitative PCR (qPCR) using the TB Green Advantage qPCR Premix (Catalog no. 639676; Takara Bio USA, Mountain View, CA) and the following thermocycling conditions:

| Temperature | Duration | Number of cycles |
|---|---|---|
| 95° C. | 30 sec | 1 |
| 95° C. | 5 sec | 40 |
| 60° C. | 30 sec | |

Quantitative assessment of DNA was performed by comparing the DNA concentration determined by amplifying the short fragment in a qPCR assay to a standard curve made of DNA standards at 100 pg/μL, 10 pg/μL, 1 pg/μL, and 0.1 pg/μL. Qualitative assessment of DNA was performed by using the ratio of the long fragment concentration to the short fragment concentration. This score varied from 0-1, with values approaching zero indicating greater degrees of DNA fragmentation or lower DNA integrity in the sample.

Fragmented DNA designed to mimic fragmented cfDNA protect by 1, 2, or 3 nucleosomes were prepared by mechanical shearing and size selection of gDNA. These DNA models had an average size of 180 bp, 360 bp, and 540 bp fragments respectively (FIG. 15, panel A). These fragmented DNA samples were tested using the DNA quantitation and fragmentation kit (Takara Bio USA, Mountain View, CA). This chemistry accurately measured the DNA concentration, compared to DNA concentration taken by Qubit dsDNA HS Assay Kit (FIG. 15, panel B), and accurately quantitated the level of DNA fragmentation, expressed as the DNA Fragmentation Score, for each DNA sample (FIG. 15, panel C).

### Example 7: Qualitative and Quantitative Assessment of cfDNA from Spent Media

The DNA Quantification and Fragmentation Kit (Takara Bio USA, Mountain View, CA) was used to perform a qualitative and quantitative assessment of cfDNA in spent media samples from euploid and aneuploid embryos collected 5, 6, or 7 days after in vitro fertilization. The media was exchanged before the final extraction on Day 4 for all samples. The average cfDNA concentration measured from the spent media was 1.11 pg/μL across all samples tested. There was no significant difference in the concentration or fragmentation of cfDNA released by euploid and aneuploid embryos (FIG. 16, panel A). When comparing cfDNA collected between collection days, there was a significant increase in the cfDNA concentration from Day 5 to Day 7, while there was no difference in the quality of DNA as shown by similar fragmentation scores across collection days (FIG. 16, panel B).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention are embodied by the appended claims.

1. A method of preparing nucleic acid molecules from a sample, the method comprising:
a. optionally lysing the sample to release the nucleic acid molecules;
b. isolating the released nucleic acid molecules using a plurality of beads for binding of the nucleic acid molecules to the plurality of beads;
c. adding one or more reagents for performing a nucleic acid assay to the bound nucleic acid molecules.

2. The method of claim 1, wherein at least a portion of the bound nucleic acid molecules are eluted in the one or more reagents.

3. The method of claim 1, further comprising performing steps a-c in a single vessel.

4. The method of claim 1, wherein one or more of steps a-c are performed simultaneously.

5. The method of claim 1, wherein the sample is lysed using a lysis reagent.

6. The method of claim 5, wherein the lysis reagent comprises a protease.

7. (canceled)

8. The method of claim 1, wherein the nucleic acid assay is a polymerase chain reaction, whole genome amplification, targeted sequencing, whole transcriptome amplification, sequencing, or any combination thereof.

9. The method of claim 1, wherein the nucleic acid assay is performed for determining at least one genetic variant.

10. The method of claim 9, wherein the at least one genetic variant is aneuploidy, mosaicism, single nucleotide polymorphism, and any combination thereof.

11. (canceled)

12. The method of claim 1, wherein the plurality of beads are magnetic beads.

13. (canceled)

14. (canceled)

15. The method of claim 1, wherein the sample is selected from the group consisting of blood, whole blood, plasma, serum, urine, cerebrospinal fluid, feces, saliva, sweat, tears, pleural fluid, pericardial fluid, peritoneal fluid, semen, uterine lavage, breast milk, extracellular vesicles, culture media, somatic cells, germ cells, fetal cells, pap smear, maternal cells, and environmental sample.

16. A method of preparing nucleic acid molecules from a sample, the method comprising:
a. lysing the sample to release the nucleic acid molecules;
b. isolating the released nucleic acid molecules using a plurality of beads for binding of the nucleic acid molecules to the plurality of beads;
c. eluting the bead-bound nucleic acid molecules from the plurality of beads directly into reagents for performing a nucleic acid assay on the released nucleic acids while the beads remain present in the reaction mixture.

17. The method of claim 16, further comprising performing steps a-c in a single vessel.

18. The method of claim 16, wherein one or more of steps a-c are performed simultaneously.

19. The method of claim 16, wherein the lysis in step a) is performed using a protease.

20. (canceled)

21. The method of claim 16, wherein the nucleic acid assay is a polymerase chain reaction, whole genome amplification, targeted sequencing, whole transcriptome amplification, sequencing, or any combination thereof.

22. The method of claim 16, wherein the nucleic acid assay is performed for determining at least one genetic variant.

23. The method of claim 22, wherein the at least one genetic variant is aneuploidy, mosaicism, single nucleotide polymorphism, and any combination thereof.

24. (canceled)

25. The method of claim 16, wherein the plurality of beads are magnetic beads.

26. (canceled)

27. (canceled)

28. The method of claim 16, wherein the sample is selected from the group consisting of blood, whole blood, plasma, serum, urine, cerebrospinal fluid, feces, saliva, sweat, tears, pleural fluid, pericardial fluid, peritoneal fluid, semen, uterine lavage, breast milk, extracellular vesicles, culture media, somatic cells, germ cells, fetal cells, pap smear, maternal cells, and environmental sample.


SEQUENCE LISTING (4 sequences)

SEQ ID NO: 1 - DNA, 20 bp, Artificial sequence
  Synthetic sequence
cctgaggtcaggagttcgag

SEQ ID NO: 2 - DNA, 20 bp, Artificial sequence
  Synthetic sequence
cccgagtagctgggattaca

SEQ ID NO: 3 - DNA, 19 bp, Artificial sequence
  Synthetic sequence
gtggctcacgcctgtaatc

SEQ ID NO: 4 - DNA, 18 bp, Artificial sequence
  Synthetic sequence
caggctggagtgcagtgg